(19) United States
(12) Patent Application Publication
(10) Pub. No.: US 2022/0254492 A1
(43) Pub. Date: Aug. 11, 2022

(54) SYSTEM AND METHOD FOR AUTOMATED DETECTION OF CLINICAL OUTCOME MEASURES

(71) Applicant: Bios Health Ltd., Cambridge, Cambridgeshire (GB)

(72) Inventors: Oliver ARMITAGE, Cambridge (GB); Emil HEWAGE, Aberdeen, Aberdeenshire (GB); Tristan EDWARDS, Oxfordshire (GB); Susannah LEE, Andover (GB); Lorenz WERNISCH, Cambridge (GB); Matjaz JAKOPEC, Cambridge (GB); Bret PATTERSON, Cambridge (GB); Catherine HANLEY, Swardeston Norfolk (GB)

(21) Appl. No.: 17/622,673

(22) PCT Filed: Jun. 26, 2020
(86) PCT No.: PCT/GB2020/051561

§ 371 Date: Dec. 23, 2021

(30) Foreign Application Priority Data
Jun. 26, 2019 (GB) 1909211.3

(51) Int. Cl. G16H 50/20

(52) U.S. Cl. CPC G16H 50/20

(57) ABSTRACT

Systems, apparatus, and method(s) are provided for automatically detecting and/or estimating one or more clinical biomarker(s) of a subject. Sensor data is received from one or more sensor(s) associated with the subject. The sensor data is using a first set of machine learning (ML) model(s) configured to extract portions of sensor data relevant for estimating one or more clinical biomarker(s) of the subject. The extracted portions of sensor data from sensors associated with the subject are processed to determine one or more clinical biomarkers. The processing of the received extracted portions of sensor data includes using a second set of ML model(s) configured to estimate the one or more clinical biomarker(s) of the subject. The portions of sensor data may comprise the first set of ML model(s) being configured to extract segments of sensor data and classify the segments of sensor data based on one or more clinical biomarker components for use in the estimation of one or more clinical biomarkers of interest. The second set of ML model(s) are configured to estimate the one or more clinical biomarker(s) of the subject based on the extracted and classified segments of sensor data.

The present application relates to a system, apparatus and method for automated detection and/or estimation of clinical outcome measures and/or clinical biomarkers.

### BACKGROUND

Clinical biomarkers, also referred to as objective outcome measures (OOMs) or clinical outcome measures, of a patient or subject are used to quantitatively indicate the patient's or subject's condition in a particular body system or body part of the patient or subject. The subject or patient may comprise or represent any organism or biological life that may include, by way of example only but is not limited to, humans, animals, and the like. The terms subject or patient are used herein interchangeably. Clinicians, doctors and researchers may use one or more clinical biomarkers of a subject for assisting in forming the basis of clinical decisions, diagnosing conditions, and/or proposing suitable treatments of any underlying conditions of the patient or subject indicated by the clinical biomarkers. Clinical biomarkers are influential in the diagnosis of a condition, selection of the type of treatment for that condition and/or treatment costs.

A clinical biomarker may comprise or represent data representative of a metric or value that is calculated from a subject performing or undergoing a specific test in a clinical or laboratory environment that can be used as an indicator of a particular disease state or some other biological or physiological state of the subject. Examples of a clinical biomarker of a patient or subject may include, by way of example only but are not limited to, OOM(s), biomarkers and the like. Some example of clinical biomarkers include, by way of example only but are not limited to, Timed Up and Go (TUG) for overall mobility; frequency of urinary voiding for incontinence; and seizure severity for epilepsy.

Conventionally, clinical biomarkers are determined from measurements of the patient or subject performing specific clinical tests, each of which correspond to estimating or calculating one or more particular clinical biomarkers of the subject. Typically, the patient or subject is required to travel to a clinical or laboratory environment and perform these specific clinical tests. Specialist personnel (e.g. clinicians, doctors, researchers, technicians or scientists) may instruct the patient to perform one or more specific clinical tests corresponding to one or more clinical biomarkers. During the tests, the specialist personnel perform the required medical measurements of the subject manually under very high degrees of observation and control.

In addition, such clinics and/or laboratories are very specialised environments with specialist instruments for measuring the necessary clinical biomarkers associated with each specific clinical test. However, not all clinics and/or laboratories may have the range of specialist instruments for a patient or subject to perform all of the required specific clinical tests during diagnosis/treatment of a condition. This means patients or subjects may be required to travel between different clinics/laboratories. This further wastes time and resources that may be better spent treating, rehabilitating, and/or maintaining the comfort of the patient or subject.

In essence, estimating clinical biomarkers requires, by way of example only but is not limited to, at least the following: a) time of specialist personnel, which can be very limited; b) patient or subject trips to/from a clinic/laboratory for measurement of one or more clinical biomarkers, which limits the frequency of such measurements; and c) expensive/high cost of specialised equipment and controlled environments for ensuring the patient performs the required test(s) correctly and ensuring specialist personnel can take the required measurements and/or observations of the patient or subject during the test(s).

It is desirable to be able to perform measurements of clinical biomarkers of a patient or subject anywhere, anytime and/or in real time in order to provide improved effectiveness and overcome one or more of the above-mentioned disadvantages as described above.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter.

The present disclosure provides apparatus, system(s) and/or method(s) for automated detection, estimation and/or calculation of clinical biomarker(s) or clinical outcome measure(s) using ML models configured to analyse sensor data taken continuously, periodically or aperiodically or over a time interval from one or more sensors associated with a subject or patient when the subject or patient is at least performing everyday activities (or daily activities) associated with their normal life and/or during specialist clinical tests associated with one or more clinical biomarkers, and the ML model(s) configured for identifying, extracting and classifying portions, segments or sub-components of the sensor data useful for estimating one or more clinical biomarkers of the subject. The portions, segments or sub-components of the sensor data are further analysed and/or processed using further ML models and/or mathematical models configured to estimate one or more clinical biomarker(s) of the subject based on the identified, extracted and/or classified portions, segments or sub-components of the sensor data. Estimates of clinical biomarkers are constructed and calculated using ML models operating on one or more sensor datasets associated with the subject. The estimated/detected clinical biomarkers that are output may be stored, sent and/or displayed to at least the specialist personnel (e.g. clinicians, doctors, researchers, scientists and the like) and/or the subject for assisting with further analysis or diagnosis of the condition of the subject, recommendation and/or selection of suitable health regimes, treatments and/or type of treatment for treating the condition of the subject.

In a first aspect, the present disclosure provides a computer-implemented method for detecting and/or extracting portions of sensor data for estimating one or more clinical biomarker(s) of a subject comprising: receiving sensor data from a plurality of sensors associated with the subject; processing the sensor data by inputting the sensor data to a first set of ML model(s) configured to extract portions of sensor data relevant for constructing and estimating clinical biomarker(s) of the subject; outputting the extracted portions of sensor data for input to a second set of ML model(s) configured to construct and estimate one or more clinical biomarker(s) of the subject.

Preferably, the computer-implemented method further comprising estimating clinical biomarker(s) of the subject based on inputting the extracted portions of sensor data to the second set of ML model(s) configured to estimate the one or more clinical biomarker(s) of the subject.

In a second aspect, the present disclosure provides a computer-implemented method for estimating clinical biomarkers of a subject, the method comprising: receiving portions of sensor data extracted from sensors associated with the subject, wherein the portions of sensor data are extracted using a first set of ML model(s) configured for extracting the relevant portions of sensor data for constructing and estimating one or more clinical biomarkers of the subject; and processing the extracted portions of sensor data using a second set of ML model(s) configured to estimate one or more clinical biomarkers of a subject.

Preferably, the computer-implemented method further comprising extracting portions of sensor data based on inputting the sensor data to the first set of ML model(s) configured to extract portions of sensor data relevant for constructing and estimating clinical biomarkers of the subject.

In a third aspect, the present disclosure provides a computer-implemented method for estimating one or more clinical biomarker(s) of a subject, the method comprising: receiving sensor data from one or more sensor(s) associated with the subject; processing the sensor data using a first set of machine learning (ML) model(s) configured to extract portions of sensor data relevant for estimating one or more clinical biomarker(s) of the subject; receiving the extracted portions of sensor data from sensors associated with the subject; and processing the received extracted portions of sensor data using a second set of ML model(s) configured to estimate the one or more clinical biomarker(s) of the subject.

Preferably, the computer-implemented method of the first, second and/or third aspects, wherein the sensor data comprises real-time sensor measurements of the subject.

Preferably, the computer-implemented method of the first, second and/or third aspects, wherein the sensor data comprises sensor measurements of the subject taken whilst the subject performs their everyday activities.

Preferably, the computer-implemented method of the first, second and/or third aspects, wherein the sensor data comprises sensor measurements of the subject taken from a device recording data during administration of a treatment to the subject.

Preferably, the computer-implemented method of the first, second and/or third aspects, wherein the sensor data comprises sensor measurements of the subject taken continuously whilst the subject performs their everyday activities, the method further comprising: pre-processing the sensor data using the first set of ML model(s) for extracting and classifying portions of the sensor data, and transmitting the extracted and classified portions of sensor data to a second computing device or unit for estimating, using the second set of ML model(s), one or more clinical biomarkers of interest present in the extracted and classified portions of sensor data.

Preferably, the computer-implemented method of the first, second and/or third aspects, wherein the portions of sensor data extracted by the first set of ML model(s) are further processed by one or more pre-processing algorithm(s) or further ML model(s) prior to inputting the extracted portions of sensor data to the second set of ML model(s) configured to estimate the one or more clinical biomarker(s) of the subject.

Preferably, the computer-implemented method of the first, second and/or third aspects, wherein a clinical biomarker comprises data representative of a metric or value that is calculated by a subject performing or undergoing a specific test in a clinical or laboratory environment that can be used as an indicator of a particular disease state or some other physiological state of a subject.

Preferably, the computer-implemented method of the first, second and/or third aspects, comprising constructing an estimate of the clinical biomarker based on: receiving sensor data comprising sensor measurements taken of the subject whilst the subject performs their everyday activities; extracting segments of the sensor data using the first set of ML model(s) to identify and classify each relevant segment of the sensor data based on one or more clinical biomarker components associated with the clinical biomarker; constructing an estimate of the clinical biomarker based on inputting the extracted segments associated with the clinical biomarker components into one or more of the second set of ML model(s) for estimating the clinical biomarker.

Preferably, the computer-implemented method of the first, second and/or third aspects, wherein the second set of ML model(s) estimate a set of biomarker(s) and the step of constructing an estimate of the clinical biomarker further comprises estimating the clinical biomarker based on combining the set of biomarker(s) using a mathematical model and/or one or more ML model(s) of the second set of ML model(s) configured for estimating the clinical biomarker.

Preferably, the computer-implemented method of the first, second and/or third aspects, wherein the first set of machine learning (ML) model(s) are configured to classify the extracted portions of sensor data based on one or more clinical biomarker components associated with the one or more clinical biomarker(s).

Preferably, the computer-implemented method of the first, second and/or third aspects, wherein the second set of ML models are configured to estimate one or more clinical biomarkers of the subject based on receiving the extracted portions of sensor data and corresponding one or more clinical biomarker components as input.

Preferably, the computer-implemented method of the first, second and/or third aspects, wherein one or more ML model(s) of the second set of ML models are further configured to estimate one or more further biomarkers or further clinical biomarkers based on receiving one or more estimated clinical biomarker(s) from others of the second set of ML model(s).

Preferably, the computer-implemented method of the first, second and/or third aspects, wherein the sensor data is unstructured sensor data, the method further comprising inputting the unstructured sensor data to the first set of ML model(s) for extracting portions of unstructured sensor data relevant for estimating the one or more clinical biomarker(s).

Preferably, the computer-implemented method of the first, second and/or third aspects, wherein the portions of sensor data comprise a plurality of segments of interest of the unstructured sensor data, the plurality of segments of interest associated with estimating one or more clinical biomarker(s) of interest.

Preferably, the computer-implemented method of the first, second and/or third aspects, wherein one or more of the first set of ML model(s) classify the portions of sensor data to form a labelled set of sensor data associated with one or more clinical biomarker component(s) for input to the second set of ML model(s).

Preferably, the computer-implemented method of the first, second and/or third aspects, wherein the portions of sensor data are input to the second set of ML model(s) for estimating one or more clinical biomarker(s) of interest.

Preferably, the computer-implemented method of the first, second and/or third aspects, wherein one or more of the first set of ML model(s) classify the portions of sensor data to form a first biomarker dataset associated with the subject for input to one or more of the second set of ML model(s) for estimating further clinical biomarker(s) associated with the biomarker dataset of the subject.

Preferably, the computer-implemented method of the first, second and/or third aspects, wherein the biomarker dataset of the subject comprises a set of biomarkers functionally useful as clinical biomarker components, wherein a clinical biomarker is estimated based on one or more of the clinical biomarker components.

Preferably, the computer-implemented method of the first, second and/or third aspects, wherein the biomarker dataset comprises a plurality of clinical biomarker component labels identifying the clinical biomarker components and the corresponding one or more portions of sensor data associated with each clinical biomarker component.

Preferably, the computer-implemented method of the first, second and/or third aspects, wherein each of the clinical biomarker component labels is associated with one or more portions of sensor data, the one or more portions of sensor data classified as being associated with the corresponding clinical biomarker component.

Preferably, the computer-implemented method of the first, second and/or third aspects, wherein the method further comprises training one or more of the first set of ML model(s) for extracting portions of sensor data associated with estimating one or more clinical biomarker(s) of the subject based on a first labelled sensor training dataset associated with the one or more clinical biomarker(s).

Preferably, the computer-implemented method of the first, second and/or third aspects, wherein the first labelled sensor training dataset is generated based on: receiving sensor data from sensors associated with a test subject in a bodily state associated with clinical biomarker(s) of interest; storing the sensor data for use in training the first set of one or more ML model(s); tracking the bodily state of the test subject, wherein each different bodily state defines a different clinical biomarker component for use in estimating a clinical biomarker of the subject; labelling, for each of the sensors associated with the test subject, each segment of sensor data of the test subject with a clinical biomarker component corresponding to the tracked bodily state determined for said each segment of sensor data; storing the labelled sensor datasets for use in training one or more ML techniques to generate one or more ML model(s) configured to extract portions of sensor data of interest from received sensor data and/or classify each of the extracted portions of sensor data based on the clinical biomarker component(s).

Preferably, the computer-implemented method of the first, second and/or third aspects, wherein a bodily state of the subject comprises an indication of the biological or physiological state of the subject at a specific time.

Preferably, the computer-implemented method of the first, second and/or third aspects, wherein the bodily state of the subject changes based on one or more activities performed by the subject.

Preferably, the computer-implemented method of the first, second and/or third aspects, wherein training the one or more of the first set of ML model(s) further comprises: retrieving the stored sensor data of one or more test subjects and the corresponding labelled sensor data segments associated with the sensor data; inputting the stored sensor data to a first set of ML technique(s) for training the first set of ML model(s) for outputting an indication of extracted sensor data segments and a classification of each segment in relation to one or more clinical biomarker component(s); updating the ML technique(s) based on comparing the output indication of extracted sensor data segments and clinical biomarker component classification with the corresponding segments of the labelled sensor datasets; repeating the steps of inputting and updating until the one or more ML technique(s) are determined to be validly trained; outputting the corresponding trained ML model(s) configured for use in extracting sensor data segments and classifying each segment in relation to clinical biomarker components for use in estimating one or more clinical biomarkers.

Preferably, the computer-implemented method of the first, second and/or third aspects, wherein the first labelled sensor training dataset comprises a plurality of portions of sensor data, each portion of sensor data associated with a label associated with estimating one or more clinical biomarker(s) or clinical biomarker component(s).

Preferably, the computer-implemented method of the first, second and/or third aspects, wherein the method further comprises training one or more of the second set of ML model(s) for estimating one or more clinical biomarker(s) of the subject based on a second labelled sensor dataset corresponding to estimates of corresponding one or more clinical biomarkers in relation to the labelled sensor dataset.

Preferably, the computer-implemented method of the first, second and/or third aspects, wherein the second labelled sensor dataset comprises the first labelled sensor dataset(s) and further labelled with corresponding estimate(s) of clinical biomarker(s) calculated in relation to the first labelled sensor datasets.

Preferably, the computer-implemented method of the first, second and/or third aspects, wherein the second labelled sensor training dataset is generated based on: retrieving the first labelled sensor dataset of a test subject; calculating one or more clinical biomarker(s) required to be estimated using one or more ML models based on the first labelled sensor dataset of the test subject and corresponding clinical biomarker components; labelling one or more segments of the first labelled sensor dataset with the corresponding calculated clinical biomarker(s); storing the labelled sensor datasets as the second labelled sensor datasets for use in training one or more ML techniques to generate one or more ML model(s) configured to estimate one or more corresponding clinical biomarker(s) of interest from received extracted segments of sensor data, each of which have been classified based on one or more clinical biomarker component(s).

Preferably, the computer-implemented method of the first, second and/or third aspects, wherein training the one or more of the first set of ML model(s) further comprises: retrieving the stored first and second labelled sensor datasets of one or more test subjects; inputting the first labelled sensor dataset to a second set of ML technique(s) for training the second set of ML model(s) for estimating one or more clinical biomarker(s); updating the second set of ML technique(s) based on comparing the estimated clinical biomarker(s) with corresponding clinical biomarkers of the second labelled dataset; repeating the steps of inputting and updating until the one or more ML technique(s) are determined to be validly trained; outputting the corresponding trained ML model(s) configured for use in estimating one or more clinical biomarker(s).

Preferably, the computer-implemented method of the first, second and/or third aspects, wherein the method further comprises training one or more of the second set of ML model(s) for estimating one or more clinical biomarker(s) based on one or more other clinical biomarker(s), biomarker(s), and/or associated extracted portions of sensor data.

Preferably, the computer-implemented method of the first, second and/or third aspects, the method further comprising estimating a further clinical biomarker based on a combination of one or more of the estimated clinical biomarkers.

Preferably, the computer-implemented method of the first, second and/or third aspects, wherein the combination of one or more of the estimated clinical biomarkers further comprises inputting the one or more estimated clinical biomarker(s) and/or clinical biomarker component(s) into a mathematical model for estimating the further clinical biomarker of the subject.

Preferably, the computer-implemented method of the first, second and/or third aspects, wherein the one or more sensor(s) associated with the subject comprise at least one from the group of: one or more sensor(s) internal to the body of the subject; one or more sensor(s) external to the body of the subject; one or more sensor(s) for chronic recording of one or more body systems of the subject; and one or more sensor(s) as part of a device delivering treatment to the body of a subject.

Preferably, the computer-implemented method of the first, second and/or third aspects, wherein the one or more sensor(s) associated with the subject comprise one or more sensor(s) from the group of: sensor(s) for peripheral or central neural recording; accelerometer sensor(s); gyroscope sensor(s); magnetometer sensor(s); activity sensor(s); electrocardiography (ECG) sensor(s); electrocorticography (ECoG) sensor(s); electroencephalography (EEG) sensor(s); electromyography (EMG) sensor(s); blood pressure sensor(s); blood vessel dilation sensor(s); blood vessel or cardiac volumetric sensor(s); flow rate sensor(s); blood viscosity sensor(s); dermal glucose sensor(s); oxygen content sensor(s); airway pressure sensor(s); breathing rate sensor(s); blood glucose sensor(s); intrapleural pressure sensor(s); bladder pressure sensor(s); galvanic skin resistance sensor(s); heart rate sensor(s); inflammatory marker sensor(s); temperature sensor(s); electrolytic concentration sensor(s); and any sensor for measuring biological or physiological state or conditions of the subject.

Preferably, the computer-implemented method of the first, second and/or third aspects, wherein the first set of ML model(s) are based on one or more ML methods or techniques from the group of: linear regression; logistic regression; random forest; neural network (NN); NN variants based on one or more of: autoencoders, long short-term memory, convolutional neural networks, any other NN structure for use in extracting portions of sensor data of a subject for determining clinical biomarkers of the subject; k-nearest neighbours; k-means; support vector machines; Naïve Bayes classifier; principal component analysis; AdaBoost; gradient boosting; Gaussian processes; any other ML method or technique for use in extracting relevant portions of sensor data of a subject for determining clinical biomarkers of the subject.

Preferably, the computer-implemented method of the first, second and/or third aspects, wherein the second set of one or more ML model(s) are based on one or more ML methods or techniques from the group of: linear regression; logistic regression; random forest; neural network (NN); NN variants based on one or more of: autoencoders, long short-term memory, convolutional neural networks, any other NN structure for use in processing portions of sensor data of a subject for determining clinical biomarkers of the subject; k-nearest neighbours; k-means; support vector machines; Naïve Bayes classifier; principal component analysis; AdaBoost; gradient boosting; Gaussian processes; any other ML method or technique configured for use in processing portions of sensor data of a subject for determining clinical biomarkers of the subject; and/or any other ML technique for generating an ML model for extracting and/or classifying sensor data segments and/or estimating clinical biomarker(s) therefrom.

Preferably, the computer-implemented method of the first, second and/or third aspects, wherein the clinical biomarker comprises at least one or more from the group of: an objective outcome measure of the subject; an indication of the physiological state of the subject; an indication of the biological state of the subject; an indication of the state associated with a bodypart of the subject; an indication of the status or condition associated with a bodypart of the subject; an indicator of a particular physiological state of a bodypart of the subject; an indication of the subject performing a physiological activity; a clinical biomarker of the subject derived from one or more specific specialist clinical tests and/or sensor data associated with the subject; and a clinical biomarker of the subject derived from one or more other clinical biomarker(s) and/or sensor data associated with the subject.

Preferably, the computer-implemented method of the first, second and/or third aspects, wherein a clinical biomarker component comprises an indication of at least one or more physiological activities from the group of: an indication of the subject walking; an indication of the subject standing; an indication of the subject sitting; an indication of the subject transitioning from sitting to standing; an indication of the subject transitioning from standing to standing; an indication of the subject turning; an indication of the subject falling; an indication of the subject stumbling; an indication of the subject running; an indication of the subject climbing stairs; and an indication of the subject performing any other physiological activity.

Preferably, the computer-implemented method of the first, second and/or third aspects, wherein a clinical biomarker comprises an objective outcome measure of the subject from at least one or more of the group of: an indication of walk time of the subject; an indication of time-up-and-go-score or metric of the subject; a risk score associated with a physiological activity of the subject; a health score associated with cardiovascular health of the subject; a risk score associated with a neurological activity of the subject; and any other score, indication or metric associated with subject.

Preferably, the computer-implemented method of the first, second and/or third aspects, wherein a clinical biomarker comprises data representative of a physiological state of a bodypart of the subject based on one or more from the group of: a heart rate of the subject; a blood pressure of the subject; a baroreceptor sensitivity of the subject; a heart rate variability of the subject; one or more stroke event(s) of the subject; one or more body part state phases of the subject; one or more urination event(s) of the subject; one or more seizure events of the subject; one or more neurological event(s) of the subject; and any other event, measurement or phase associated with a body part of the subject.

According to a fourth aspect of the invention, there is provided a computer-implemented method for training a first set of ML models for extracting and classifying portions of sensor data in relation to clinical biomarker components of a subject for estimating one or more clinical biomarkers of the subject, the method comprising: retrieving sensor data of one or more test subjects and the corresponding labelled sensor training dataset comprising sensor data segments associated with the sensor data, wherein the labelled sensor data segments are labelled based on a set of clinical biomarker components; inputting sensor data to a first set of ML technique(s) for training a first set of ML model(s) for outputting an indication of extracted sensor data segments and a classification of each segment in relation to one or more clinical biomarker component(s); updating the first set of ML technique(s) based on comparing the output indication of extracted sensor data segments and clinical biomarker component classification with the corresponding labelled sensor data segments; repeating the steps of inputting and updating until the ML technique(s) are determined to be validly trained; outputting the corresponding trained first set of ML model(s) configured for extracting sensor data segments and classifying each extracted sensor data segment in relation to clinical biomarker components for use in estimating one or more clinical biomarkers.

Preferably, the labelled sensor training dataset is generated based on: receiving sensor data from sensors associated with a test subject performing activities associated with clinical biomarker(s) of interest; storing the sensor data for use in training the first set of one or more ML model(s); tracking the type of activity the test subject performs, wherein each type of activity defines a different clinical biomarker component for use in estimating a clinical biomarker; labelling, for each of the sensors associated with the test subject, each segment of sensor data of the test subject with a clinical biomarker component corresponding to the tracked activity type determined for said each segment of sensor data; storing the labelled sensor datasets for use in training one or more ML techniques to generate one or more ML model(s) configured to extract portions of sensor data of interest from received sensor data and/or classify each of the extracted portions of sensor data based on the clinical biomarker component(s).

Preferably, training the one or more of the first set of ML model(s) further comprises: retrieving the stored sensor data of one or more test subjects and the corresponding labelled sensor data segments associated with the sensor data; inputting the stored sensor data to a first set of ML technique(s) for training the first set of ML model(s) for outputting an indication of extracted sensor data segments and a classification of each segment in relation to one or more clinical biomarker component(s); updating the ML technique(s) based on comparing the output indication of extracted sensor data segments and clinical biomarker component classification with the corresponding segments of the labelled sensor datasets; repeating the steps of inputting and updating until the one or more ML technique(s) are determined to be validly trained; outputting the corresponding trained ML model(s) configured for use in extracting sensor data segments and classifying each segment in relation to clinical biomarker components for use in estimating one or more clinical biomarkers.

Preferably, the first labelled sensor training dataset comprises a plurality of portions of sensor data, each portion of sensor data associated with a label associated with estimating one or more clinical biomarker(s) or clinical biomarker component(s).

According to a fifth aspect, there is provided a computer-implemented method for training a set of ML models for estimating one or more clinical biomarkers of a subject, the method comprising: receiving a labelled sensor training dataset comprising extracted portions of sensor data classified in relation to clinical biomarker components and labelled with calculated clinical biomarkers of the subject; inputting the labelled sensor training dataset to a set of ML technique(s) for generating one or more ML model(s) for estimating one or more clinical biomarker(s) of the subject based on the labelled sensor training dataset; updating the ML technique(s) based on comparing the estimated one or more clinical biomarker(s) with the corresponding calculated clinical biomarkers of the subject; repeating the inputting and updating steps until the ML techniques are determined to be validly trained; outputting the corresponding trained set of ML model(s) configured for estimating clinical biomarkers based on sensor data segments classified to the corresponding clinical biomarker components.

Preferably, the labelled sensor training dataset is generated based on: retrieving a first labelled sensor dataset of a test subject, the first labelled sensor dataset comprising sensor data segments classified based on a set of clinical biomarker components; calculating one or more clinical biomarker(s) and/or one or more clinical biomarker components of the test subject required to be estimated using one or more ML models based on the first labelled sensor dataset of the test subject and corresponding clinical biomarker components; labelling one or more segments of the first labelled sensor dataset with the corresponding calculated clinical biomarker(s); and storing the labelled sensor training dataset for use in training one or more ML techniques to generate one or more ML model(s) configured to estimate one or more corresponding clinical biomarker(s) of interest from received extracted segments of sensor data, each of which have been classified based on one or more clinical biomarker component(s).

Preferably, the method further comprises training one or more of the second set of ML model(s) for estimating one or more clinical biomarker(s) based on one or more other clinical biomarker(s) and/or associated extracted portions of sensor data.

Preferably, the method further comprising estimating a further clinical biomarker based on a combination of one or more of the estimated clinical biomarkers.

According to a sixth aspect, there is provided a ML model obtained by implementing the computer-implemented method according to any one of the first, second, third, fourth and/or sixth aspects.

According to a seventh aspect, there is provided an apparatus comprising a processor, a memory, a communication interface, the processor coupled to the memory and communication interface, the apparatus configured to implement the computer-implemented method according to any of the first, second, third, fourth and/or sixth aspects.

According to a eighth aspect, there is provided a system for estimating one or more clinical biomarker(s) of a subject, the system comprising: a communication interface for receiving sensor data from one or more sensor(s) associated with the subject; a sensor signal pre-processing unit for extracting portions of sensor data using a first set of machine learning (ML) model(s) configured to extract said portions of the received sensor data relevant for constructing and estimating one or more clinical biomarker(s) of the subject; and a clinical biomarker estimation unit for estimating one or more clinical biomarker(s) of the subject using a second set of ML model(s) configured to estimate the one or more clinical biomarker(s) of the subject based on the extracted portions of sensor data.

Preferably, the first set of machine learning (ML) model(s) are configured to classify the extracted portions of sensor data based on one or more clinical biomarker components associated with constructing and estimating the one or more clinical biomarker(s).

Preferably, the second set of ML models are configured to construct an estimate one or more clinical biomarkers of the subject based on receiving the extracted portions of sensor data and corresponding one or more clinical biomarker components as input.

Preferably, the sensor data comprises real-time sensor measurements of the subject.

Preferably, the system of the eighth aspect where one or more of the communication interface, the sensor signal pre-processing unit, or clinical biomarker estimation unit are configured to implement the corresponding steps of the computer-implemented method according to any of the first, second, third, fourth and/or sixth aspects.

According to a ninth aspect, there is provided a system comprising an apparatus according to the seventh aspect.

According to a tenth aspect, there is provided a system comprising a processor, a memory, a communication interface, the processor coupled to the memory and communication interface, the apparatus configured to implement the computer-implemented method according to any of the first, second, third, fourth and/or sixth aspects, combinations thereof, modifications thereto, and/or features thereto.

According to a eleventh aspect, there is provided a computer-readable medium comprising computer code or instructions stored thereon, which when executed on one or more processor unit(s), causes the one or more processor unit(s) to implement the computer-implemented method according to any of the first, second, third, fourth and/or sixth aspects, combinations thereof, modifications thereto, and/or features thereto. The computer-readable medium may be a tangible computer-readable medium.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

It is desirable to be able to perform measurements of clinical biomarkers of a patient or subject anywhere, anytime and/or in real time in order to provide improved effectiveness.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a schematic diagram illustrating a first example of a system for estimating clinical biomarkers according to a first embodiment;

FIG. 1b is a more detailed diagram of the data processing pipeline of the system of FIG. 1a;

FIG. 1c is a detailed diagram of a first example of the first and second processing stages of the data processing pipeline of FIG. 1b;

FIG. 1d is a detailed diagram of a second example of the first and second processing stages of the data processing pipeline of FIG. 1b;

FIG. 2a is a flow diagram of a method for generating labelled training dataset for use in the first processing stage of the data processing pipeline of FIG. 1b;

FIG. 2b is a flow diagram of a method for training a first set of machine learning (ML) technique(s) to generate a first set of ML model(s) for use in the first processing stage of the data processing pipeline of FIG. 1b;

FIG. 2c is a flow diagram of a method for generating a further labelled training dataset for the second processing stage based on the output of the first processing stage of the data processing pipeline of FIG. 1b;

FIG. 2d is a flow diagram of a method for training a second set of ML technique(s) to generate a second set of ML model(s) for use in the second processing stage of the data processing pipeline of FIG. 1b;

FIG. 2e is a flow diagram of a method for using the first set of ML model(s) in the first processing stage of the data processing pipeline of FIG. 1b;

FIG. 2f is a flow diagram of a method for using the second set of ML model(s) in the second processing stage of the data processing pipeline of FIG. 1b;

FIG. 3a is a schematic diagram of an example of a first ML model for the first processing stage of data processing pipeline of FIG. 1b for estimating clinical biomarker(s) associated with timed up and go or fall risk;

FIG. 3b is a schematic diagram of an example of a second set of ML model(s) for the second processing stage of data processing pipeline of FIG. 1b for estimating clinical biomarker(s) associated with timed up and go or fall risk of a subject;

FIG. 4 is a schematic diagram of another example of a first ML model for use in the first processing stage of data processing pipeline of FIG. 1b and a second set of ML model(s) for use in the second processing stage of data processing pipeline of FIG. 1b for estimating clinical biomarker(s) associated with timed up and go or fall risk of a subject;

FIG. 5a is a schematic diagram of an example of a first ML model for use in the first processing stage of data processing pipeline of FIG. 1b for estimating clinical biomarker(s) associated with urination events of a subject;

FIG. 5b is a schematic diagram of an example of a second set of ML/mathematical model(s) for the second processing stage of data processing pipeline of FIG. 1b for estimating clinical biomarker(s) associated with urination events of a subject;

FIG. 6a is a schematic diagram of an example of a first ML model for use in the first processing stage of data processing pipeline of FIG. 1b for estimating clinical biomarker(s) associated with seizure events of a subject;

FIG. 6b is a schematic diagram of an example of a second set of ML model(s) for the second processing stage of data processing pipeline of FIG. 1b for estimating clinical biomarker(s) associated with seizure events of a subject;

FIG. 7a is a schematic diagram of an example computing system for estimating clinical biomarkers according to a second embodiment; and

FIG. 7b is a schematic diagram of another example system for estimating clinical biomarkers according to a third embodiment.

Common reference numerals are used throughout the figures to indicate similar features.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

It should be noted that although exemplary examples, descriptions and/or embodiments of the invention are provided in the following description. For the avoidance of any doubt, the features described in any embodiment and/or the embodiments themselves are combinable with the features of any other embodiment and/or any other embodiment unless express statement to the contrary is provided herein. Simply put, the features described herein are not intended to be distinct or exclusive but rather complementary and/or interchangeable.

The present disclosure provides a clinical biomarker estimation system using machine learning (ML) techniques for generating ML models configured to analyse sensor data taken continuously, periodically or aperiodically from one or more sensors associated with a subject or patient when the subject or patient is at least performing everyday activities (or daily activities) associated with their normal life and/or during specialist clinical tests associated with one or more clinical biomarkers, and for identifying, extracting and classifying portions, segments or sub-components of the sensor data useful for estimating one or more clinical biomarkers of the subject. The portions, segments or sub-components of the sensor data are further analysed using further ML technique(s) to generate ML models and/or mathematical models configured to estimate one or more clinical biomarker(s) of the subject. In this manner, estimates of clinical biomarkers are constructed and calculated using ML models operating on one or more sensor datasets, which may be disparate sensor datasets associated with different body parts of the subject, and extracting subcomponents of interest from the sensor datasets for inferring clinical biomarker estimates therefrom. The estimated clinical biomarkers that are output may be stored, sent and/or displayed to at least the specialist personnel (e.g. clinicians, doctors, researchers, scientists and the like) and/or subject for assisting with further analysis and/or diagnosis of the condition of the subject, recommendations and/or selection of suitable health regimes, treatments and/or type of treatment for treating the condition of the subject. Although, clinical biomarkers are typically interpretable by a clinician, specialist personnel and the like in relation to the subject, clinical biomarkers may also be shown to the clinician, specialist personnel, subject or patient in a graphical, tabular or other format. Display of clinical biomarkers may be shown relative to one or more pre-set level(s) or zones that are indicative of certain known outcomes relative to the population or relevant sub-section of the population.

The subject may be incorporated or provided with one or more sensors internal and/or external to the body or bodypart of the subject for measuring a bodily state, biological or physiological state of the subject. The bodily state of a subject may be described as, by way of example only but it not limited to, an indication of the biological or physiological state of the subject or a part or sub-system of the subject at a specific time. A bodily state may in some instances be defined by the values of a group of one or more biomarkers about the subject. A subject may have a plurality of bodily states. Sensors may be used to provide sensor data measuring components associated with one or more bodily states of the subject. Examples of other sensors that may be used that are internal or external to the body of the subject and may be placed in, on or around one or more body parts of the subject 102 for measuring components indicative of one or more bodily states of the subject include, by way of example only but is not limited to, sensors associated with peripheral or central neural recording; inertial sensors, accelerometer; gyroscope; magnetometer; electrocardiography (ECG); electrocorticography (ECoG); electroencephalography (EEG); electromyography (EMG); blood pressure; blood vessel dilation sensor(s); blood vessel or cardiac volumetric sensor(s); flow rate sensor(s); blood viscosity sensor(s); dermal glucose sensor(s); oxygen content sensor(s); airway pressure; breathing rate sensor(s); blood glucose sensor(s); blood glucose; intrapleural pressure; heart rate variability; bladder pressure; galvanic skin resistance sensor(s); heart rate; inflammatory marker sensor(s); temperature sensor(s); electrolytic concentration sensor(s) (e.g. Sodium or Potassium ion sensors); or any other sensor or sensors for measuring the characteristics or a signal associated with a body part of the subject 102. A sensor may be part of a device recording sensor measurements of the subject during administration of a treatment to the subject.

This means that sensors can be provided and used by the subject and “live” with the subject at home and/or work recording and/or analysing the sensor data associated with one or more bodily state(s), biological state(s), or physiological state(s) of the subject that enables estimation of clinical biomarkers of the subject as described herein. The sensors may be used by the subject continuously during their daily activities, or used continuously for a period of time during a daily activity, without supervision of specialist personnel such as a clinician and the like. The sensors and sensor data measured are also not subject to the same degree of control as is the case when the subject performs a specialist clinical test under supervision of a clinician. Thus, the sensor data measured and recorded of the subject comprises unstructured sensor data or signals, which have not been labelled or analysed. The clinical biomarker estimation system and methods therefore is focused on using this kind of unstructured sensor data in which ML technique(s) are used to generate ML models capable of identifying, classifying subcomponents of the sensor data during the activities of the subject and extracting the classified subcomponents based on the same kind or similar kind of metrics, or clinical biomarker components, that may be measured and extracted in a clinical setting. The sensor data that is continuously measured or taken of the subject may be long-term unstructured sensor data in which the application of ML techniques/models and where applicable one or more mathematical models, algorithm and formula, are used to infer clinical biomarkers (also known as objective outcome measures (OOMs)) from specific signals that are identified to be of interest within the sensor data taken from sensors associated with the subject.

As is widely appreciated, a biomarker is anything that can be used as an indicator of a particular disease state or some other biological and/or physiological state of a subject. A clinical biomarker (or clinical outcome measure) may comprise or represent data representative of a metric or value calculated from a subject performing or undergoing a specific test in a clinical or laboratory environment that can be used as an indicator of a particular disease state or some other biological or physiological state of the subject. A clinical biomarker is a biomarker that can be used and interpreted by a clinician or specialist personnel as the basis for making a health regime decision, treatment decision or medical diagnosis as the biomarker has been validated or otherwise accepted by a scientific and/or clinical community and the like as being a relevant metric for that disease or physiological state. As such a clinical biomarker will have an agreed test in a clinical or laboratory environment for manually calculating it. Clinical biomarkers are hence a subset of biomarkers that have been agreed for use in a medical setting by specialist and/or clinical personnel.

Examples of clinical biomarkers (or clinical outcome measures) may include, by way of example only but is not limited to, one or more from the group of: data representative of a metric or value indicating the bodily state, biological or physiological state or condition of the subject based on the subject performing a specific test in a clinical or laboratory environment; data representative of an indicator, metric or value derived from one or more qualitative tests taken by a subject; data representative of an indicator, value or metric derived from one or more quantitative tests taken of a subject; a heart rate of the subject; one or more stroke event(s) of the subject; one or more body part state phases of the subject; one or more urination event(s) of the subject; one or more seizure events of the subject; one or more neurological event(s) of the subject; and any other event or phase associated with a body part of the subject; data representative of a physiological or biological activity of the subject derived from sensor data measured from sensors associated with the subject; a clinical outcome measure of the subject; an objective outcome measure (OOM) of the subject; an indication of walk time of the subject; an indication of timed-up-and-go score of the subject; an indication of time-to-get-up of the subject; a risk score associated with a physiological activity of the subject; a risk score associated with a neurological activity of the subject; and any other score or indication associated with subject; an indication of the physiological state of the subject; an indication of the biological state of the subject; an indication of the state associated with a bodypart of the subject; an indication of the status or condition associated with a bodypart of the subject; an indicator of a particular physiological state of a bodypart of the subject; an indication of the subject performing a physiological activity; a clinical biomarker of the subject derived from one or more other clinical biomarker(s) and/or sensor data or other measurements associated with the subject; one or more clinical biomarker components that comprises or represents data representative of an indication of one or more physiological activities or sub-activities of a subject for use in determining a clinical biomarker; a clinical biomarker component from the group of: an indication of the subject walking; an indication of the subject standing; an indication of the subject sitting; an indication of the subject transitioning from sitting to standing; an indication of the subject transitioning from standing to standing; an indication of the subject turning; an indication of the subject falling; an indication of the subject running; and an indication of the subject performing any other physiological activity.

The present disclosure also provides a clinical biomarker estimation system, apparatus and methods thereto providing efficient collection and analysis of long-term sensor data that enables estimation of clinical biomarkers of a subject. The advantages of the clinical biomarker estimation system include, by way of example only but is not limited to, measuring and estimating clinical biomarkers more frequently, and more frequent analysis of the estimated clinical biomarkers by specialist personnel (e.g. clinicians and the like) more frequently resulting in higher fidelity of the everyday condition of the subject and allowing timely diagnosis to changes in the condition of a subject and/or more effective treatment; reducing, avoiding and/or eliminating specialist personnel (e.g. clinicians) dedicating time, space and specialist equipment for collecting and measuring clinical biomarkers of the subject; reducing, avoiding and/or eliminating time, transport resources, resource wastage by the subject visiting a specialist environment such as, by way of example only but not limited to, a clinic, laboratory or research centre and the like for collecting sensor data and/or sensor signals of interest are and calculating clinical biomarkers and/or interpreting thereof; allowing specialist personnel and the like to analyse and interpret the estimated clinical biomarkers without the subject being present.

Given the sensor data and number of sensors from different body parts that may contribute to the estimation or inference of a clinical biomarker, said clinical biomarker may not be directly computed from the sensor data but may be derived from a combination of subcomponents of the sensor data of the subject, or a unique combination of subcomponents of sensor data of the subject that clinicians are unable to calculate or derive. That is, the clinical biomarkers may not exist directly in the sensor data, but may be inferred or derived from the sensor data based on ML models and/or mathematical models, formula and the like.

For example, clinical biomarkers may be derived from a quantitative test such as the Timed Up & Go (TUG) clinical test of a subject that provides a TUG biomarker of the subject, which is a metric derived from a specific compound or contiguous set of motions (e.g. TUG motions) specified by the TUG clinical test. The TUG clinical test may require the TUG motions be performed contiguously by the subject under supervision of a clinician. However, the clinical biomarker estimation system does not necessarily require the subject to perform a specific clinical test, instead sensor data of the subject may be measured and collected from one or more sensors associated with the subject during the daily activities of the subject. Given this, it is likely that at no point during a day at home or work, or performing their usual daily activities, would the subject have intentionally performed the specific compound or contiguous set of TUG specialist motions. Rather, during the daily activities of the subject, the subject may have performed portions or components of the TUG set of specialist motions throughout the day, thus, a system that identifies the components of the TUG set of specialist motions from the sensor data may be able to infer the TUG clinical biomarker. The clinical biomarker estimation system uses a first set of ML model(s) for identifying, classifying and estimating sub-components, segments or portions of the long-term sensor data collected from the daily activities of the subject, and use a second set of ML model(s) based on the classified and extracted sub-components to infer or estimate a TUG clinical biomarker. Thus, the sub-components of the sensor datasets can be quantitatively found in the data and reconstructed into an estimate of a TUG clinical biomarker (e.g. a TUG score).

In another example, a clinical biomarker of a risk score of falling may be calculated by a combination of a first machine learning method that identifies portions of motion data corresponding to walking sections. A second machine learning method that characterises each section into high risk vs low risk may be used with the risk score being the number of high risk segments divided by the total number of segments.

In another example, a clinical biomarker may be derived from one or more qualitative tests like a questionnaire such as, by way of example only but not limited to, a PHQ-9 point questionnaire used to determine a PHQ-9 depression score. If a patient answers this 9 point questionnaire about their depression the summed answers are considered a clinical biomarker. Although it is apparent that the answers to the questionnaire do not exist in any raw sensor data (e.g. raw accelerometer data) from one or more sensors associated with the subject, it is possible to train a first set of one or more ML models to identify, classify, extract and calculate quantitative features from the sensor data in which a second set of one or more ML model(s) are trained to construct or infer estimates of the PHQ-9 depression score in response to the PHQ-9 questionnaire based on the quantitative features of the sensor dataset.

To illustrate a few potential embodiments of the clinical biomarker estimation system will be described based on, for simplicity and by way of example only but not limited to, to three examples of estimating or inferring clinical biomarkers based on sensor datasets of a subject. However, it is to be appreciated by the skilled person that the examples and/or exemplar clinical biomarkers described herein are not exhaustive and that the skilled person may adapt or modify the invention as described herein as the application demands. For example, the skilled person may apply the clinical biomarker estimation system, as described herein, for measuring, analysing, classifying any sensor data or signals output by a plurality of sensors associated with the subject or patient (e.g. sensors that are placed on, are external to, and/or are internal to the subject), in which the sensor data is used as described herein to infer and/or estimate any one or more clinical biomarkers using ML model(s) and/or mathematical models. In particular, it is to be appreciated by the skilled person that the invention as described herein may be adapted, modified, and/or combined with features as described herein to be configured to measure or record any sensor data from one or more sensors or a plurality of sensors associated with a subject or patient, and to identify, classify and extract sub-components or segments of the sensor data of interest using one or more ML model(s), and to estimate any one or more clinical biomarker(s) of interest of the subject using one or more ML model(s) based on the extracted sub-components, segments or portions of the sensor data for outputting the estimated or inferred clinical biomarker(s) for sending/review by specialist personnel and the like. The sensor data may be long term sensor data that is collected from a plurality of sensors associated with the subject or patient during their daily or everyday activities, in which sub-components of the sensor data are combined to form clinical biomarkers. In addition or alternatively, sensor data of a subject may be collected when a subject or patient performs one or more specialist clinical tests associated with clinical biomarkers to assist a specialist in measuring and/or determining the corresponding clinical biomarkers.

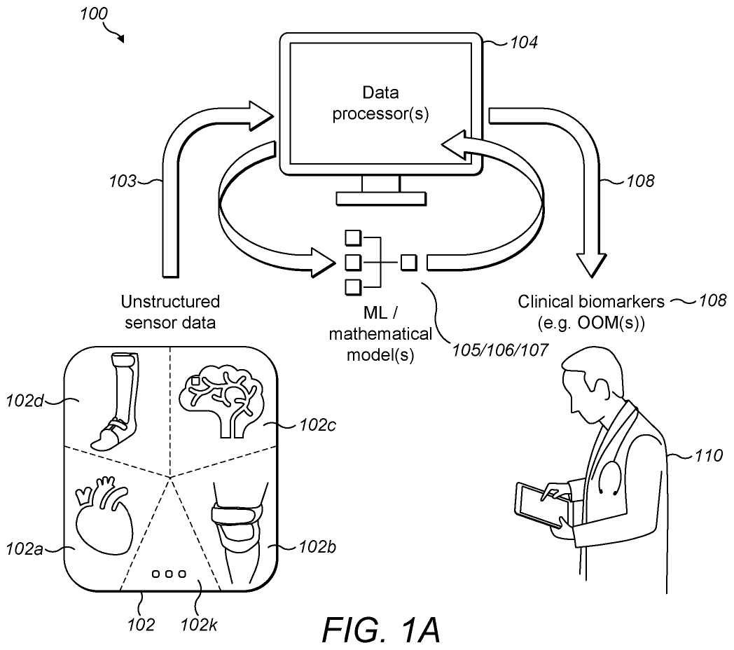

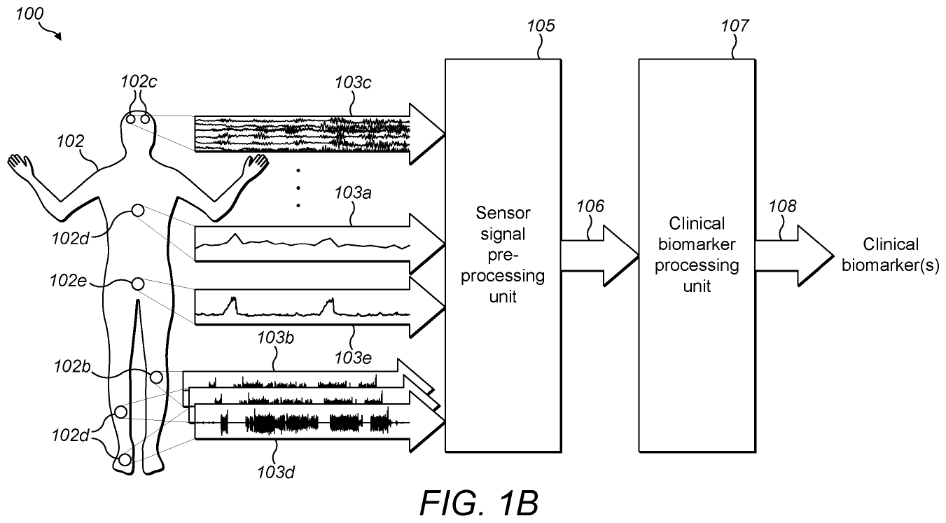

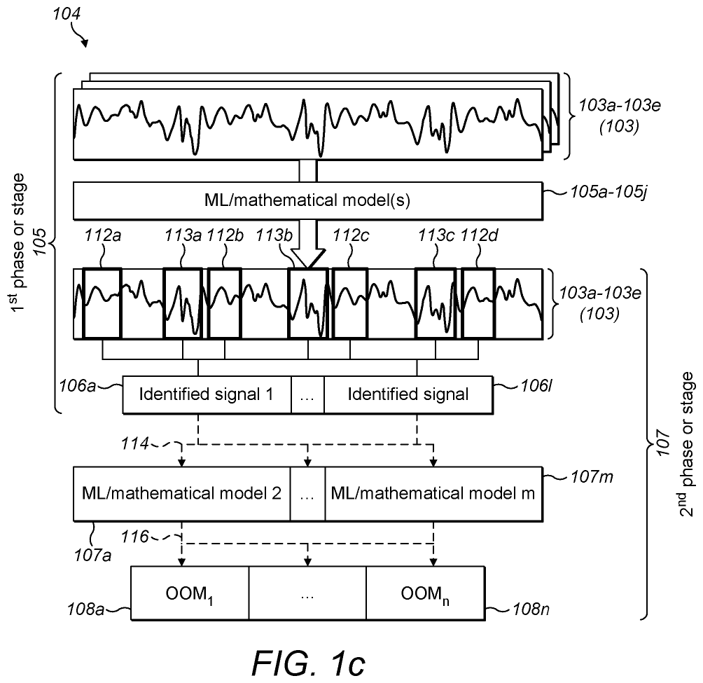

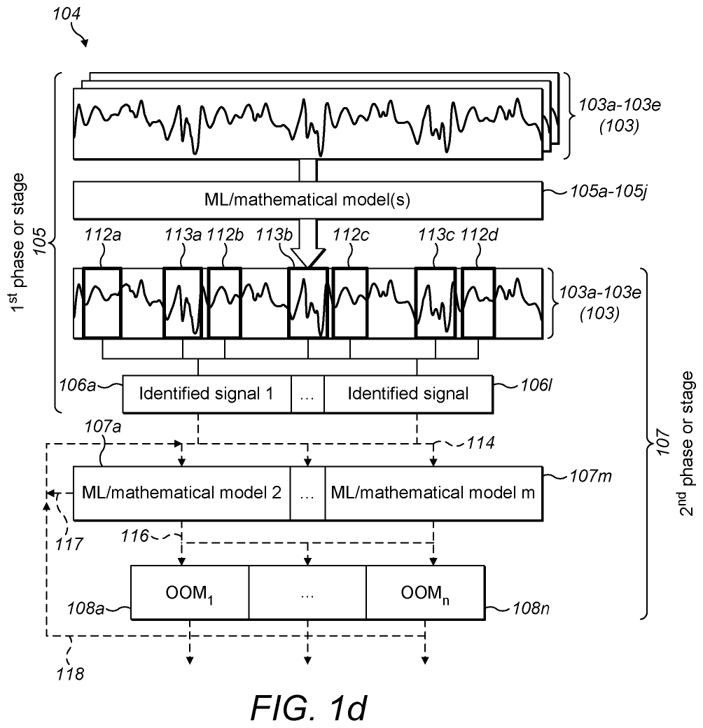

FIG. 1a is a schematic diagram illustrating a first example of a system 100 for estimating clinical biomarkers (e.g. OOM(s) or clinical outcome measures) 108 according to a first embodiment. The clinical biomarker estimation system 100 may include a data processor system 104 (computing system) for receiving sensor data or datasets recorded from one or more, or a plurality of sensors 102a-102k associated with a subject 102. The data processor system 104 may be configured to estimate or infer one or more clinical biomarker(s) of the subject 102 based on the received sensor datasets. Unstructured data are collected from the sensor(s) 102a-102k which are internal or external to the body of the subject 102. The sensor data may then be communicated to hardware of the data processing system 104 for data processing. Data processing may occur in one or more stages of data pre-processing and mathematical model application until one or more clinical biomarker(s) (e.g. OOM(s)) of interest 108 are extracted and made available for clinical analysis 110. For example, the data processing system 104 may include, by way of example only but is not limited to, one or more computational devices, local or remote to the sensor, to receive sensor data 103 and run it through one or more stages of processing. Each stage of processing may include input data pre-processing, application of a first selected ML model 105 to extract segments and/or metrics of interest from the data, and post-processing, application of a second selected ML model 107 to process the extracted segments of interest 106 for estimating or inferring clinical biomarkers and which terminates in the output of one or more clinical biomarkers 108.

One or more ML or mathematical models (or multiple ML or mathematical models) may be used to pre-process the sensor data received from the sensors 102a-102k associated with the subject 102 to determine the relevant sections of the recorded sensor data and for processing the pre-processed sensor and for inferring or estimating one or more clinical biomarkers (or corresponding multiple clinical biomarkers) from the relevant sections of recorded sensor data. Examples of ML techniques for generating one or more ML models may include or be based on, by way of example only but is not limited to, one or more from the group of: linear regression, logistic regression, random forest, neural network (and variants such as autoencoders, reinforcement learning, Long-Short-Term-Memory (LSTM), Convolutional Neural Networks (CNN), etc.), k-nearest neighbours, k-means, support vector machine, Naïve Bayes classifier, principal component analysis, AdaBoost, gradient boosting, Gaussian processes and the like; and/or any other ML technique as the application demands such as, by way of example only but not limited to, one or more supervised ML techniques, semi-supervised ML techniques, unsupervised ML techniques, linear and/or non-linear ML techniques, ML techniques associated with classification, ML techniques associated with regression and the like, modifications thereto, and/or combinations thereof. Although several ML techniques and/or ML model(s) are described herein, for simplicity and by way of example only but not limited to, for pre-processing the sensor data and for estimating clinical biomarkers based on the pre-processed sensor data, it is to be appreciated by the skilled person that any suitable ML technique and/or ML model, one or more ML technique(s) and/or ML model(s) (including mathematical model(s)), modifications and/or combinations thereof, may be used for pre-processing sensor data and/or estimating/detecting clinical biomarkers, and/or used in the clinical biomarker estimation and/or detection system 100 and/or as the application demands.

In this example, the subject or patient 102 may be fitted with a plurality of sensors 102a-102k, in which one or more of the sensors may be external to the body of the subject 102, internal to the body of the subject 102 and/or placed on the body or in the body of the subject 102. In this example, the sensors that are placed on the body of the subject 102 (e.g. are external to the body of the subject 102) may include, by way of example only but is not limited to, a heart rate sensor 102a, an knee inertial sensor 102b, and/or a foot/shin inertial sensor 102d and the like; the sensors that are placed in the body of the subject 102 (e.g. are internal to the body of the subject 102) may include, by way of example only but are not limited to, neurological transceivers/receivers and/or peripheral or central neural recording sensor(s) 102c and the like.

For example, the data processor system 104 may include several processing stages that include one or more pre-processing units and one or more clinical biomarker processing units. These are configured to form a data processing pipeline for pre-processing the received sensor data 103 from one or more sensor(s) 102a-102k, or from a plurality of sensors 102a-102k and for inferring/estimating clinical biomarkers 108 from the pre-processed sensor data 106. The pre-processing units are configured to use a first set of one or more ML models 105 trained using corresponding ML technique(s) for identifying, classifying and/or extracting sub-components, portions or segments of the sensor data of interest 106. The portions of sensor data extracted by the first set of ML model(s) 105 may be further processed by one or more pre-processing algorithm(s) or further ML model(s) prior to inputting the extracted portions of sensor data 106 to the second set of ML model(s) 107 configured to estimate the one or more clinical biomarker(s) 108 of the subject 102. The classified and extracted portions of sensor data 106 are processed by the clinical biomarker processing units using a second set of one or more ML models 107, which may also include one or more mathematical models, formula or algorithms, for estimating or inferring the one or more clinical biomarkers 108. The one or more clinical biomarkers 108 may be sent to and/or assessed by, by way of example only but is not limited to, specialist personnel (e.g. a clinician and the like) 110 and/or by a diagnostic program or apparatus and the like for assisting in, if necessary, diagnosis and/or treatment of the subject and/or patient and the like.

In another example, sensor data may be wirelessly transmitted to the data processing system 104, which may be, by way of example only but not limited to, a local or remote server, a distributed computing platform or cloud computing environment. Wireless transmission may be based on any wireless standard such as, by way of example only but not limited to, Bluetooth, Wi-Fi, 2G-5G or beyond wireless transmission, cellular data transmission and/or any other radio connection or transmission.

Alternatively in another example, the data processor system 104 may include a wearable processor on the subject 102 in which the sensor data may be transmitted by wired or wireless connection to the wearable processor on the subject 102. The wearable processor on the subject 102 may perform at least the pre-processing of the sensor data and store only the extracted and classified sensor data segments of interest prior to being processed by, by way of example only but not limited to, a local or remote server, a distributed computing platform or cloud computing environment. Should the wearable processor on the subject 102 have clinical biomarker processing units, the wearable processor may also be configured to process the pre-processed sensor data to infer or estimate the one or more clinical biomarkers of interest.

FIG. 1b is a more detailed diagram of the data processing pipeline of the data processor system 104 of FIG. 1a, where data processing occurs in one or more stages 105-107 of data pre-processing and ML model application until one or more clinical biomarkers (e.g. OOMs) of interest are extracted and made available for analysis (e.g. clinical analysis and the like). The data processing system 104 of the clinical estimation system 100 for estimating one or more clinical biomarker(s) of the subject 102 may include a first processing stage represented by a sensor signal pre-processing unit 105 and a second processing stage represented by a clinical biomarker processing unit 107. The data processing system 104 may also include a communication interface for receiving sensor data from one or more sensor(s) 102a-102d of the subject 102.

In this example, the sensor(s) associated with the subject 102 include a heart rate sensor unit 102a, a knee or leg inertial sensor unit 102b, peripheral or central neural recording sensor unit(s) 102c, and a foot/shin inertial sensor unit(s) 102d. The sensor(s) 102a-102d are used to measure measurable components of the body or body parts of the subject 102 in relation to one or more bodily state(s) of the subject 102. The heart rate sensor unit 102a may be placed on the chest of the subject 102 for measuring the heart rate of the heart body part of the subject 102 (e.g. a Polar® H10 Heart Rate Monitor). The heart rate sensor unit 102a may output heart rate sensor data 103a of the subject 102, which may vary as the subject 102 performs their daily activities, emotional activities, and/or work activities and the like. The heart rate of the subject 102 may be affected by many factors during the daily activities of the subject 102 such as, by way of example only but not limited to, movement of the subject 102, smells and/or noises experienced by the subject, touch and/or other senses experienced by the subject, emotions and/or stress experienced by the subject 102, visual stimuli received by the subject 102 and the like. The knee inertial sensor unit(s) 102b are placed on the knee body part of the subject 102 and when the subject 102 moves their knee (e.g. bending the knee, sitting, walking, standing, running etc.) they can measure the movement of the knee of the body part of the subject 102 and output knee inertial sensor data 103d, which may be used to identify metrics, values and/or parameters associated with, by way of example only but is not limited to, the subject 102 bending the knee, sitting, walking, standing, and/or running. The foot/shin inertial sensor unit(s) 102d are placed on the foot and/or shin body part of the subject 102 and when the subject 102 moves they measure the movement of the foot and/or shin body part of the subject 102 and output foot/shin inertial sensor data 103d, which may be used to identify metrics, values and/or parameters such as, by way of example only but is not limited to, the subject 102 bending the foot, sitting, walking, standing, and/or running. The urinary sensor unit(s) 102e are placed near the bladder body part of the subject 102 may detect the bladder pressure of the subject 102 and output bladder pressure sensor data 103e. The peripheral or central neural recording sensor unit(s) 102c are placed on or within the head body part of the subject 102 and output neurological data 103c. The sensor data or datasets 103a-103e from the plurality of sensors 102a-102e are received by the sensor signal pre-processing unit 105.

The sensor signal pre-processing unit 105 is configured for extracting portions of the received sensor data using a first set of machine learning (ML) model(s) that are trained or adapted/configured to extract portions of the received sensor data relevant for constructing and estimating one or more clinical biomarker(s) of the subject. The first set of ML model(s) may include a plurality of ML model(s), each of which may be configured to identify, classify and/or extract the relevant segments/sub-components of the sensor data of the corresponding sensor that have been determined to be useful for estimating one or more clinical biomarkers. The relevant segments/sub-components of the sensor data may include those portions of the sensor data that correspond to biomarkers, intermediate biomarkers or clinical biomarker components (e.g. biomarkers that may be used in the estimation, calculation and/or determination of one or more clinical biomarkers), and/or other biomarkers that may function as clinical biomarker components, which may be used or combined for estimating one or more clinical biomarker(s) of interest.

For example, the first set of ML model(s) may include, by way of example only but is not limited to, one or more of the following: a heart rate ML model may be configured for extracting and classifying relevant segments/sub-components of the heart rate sensor data 103a of interest; one or more inertial movement ML models may be configured for extracting and classifying relevant segments/sub-components of the knee, shin/foot sensor data or datasets 103b or 103d of interest; one or more neurological ML models may be configured for extracting and classifying relevant segments/sub-components of the neurological sensor data or datasets 103c of interest; one or more neurological ML models may be configured for extracting and classifying relevant segments/sub-components of the neurological sensor data or datasets 103c of interest. The extracted and classified segments of the sensor data from the sensor(s) 102a-102e forms a set of extracted and classified sensor data 106 that may correspond to biomarker dataset, which may be a set of biomarkers functionally useful as clinical biomarker components in which one or more clinical biomarker(s) may be estimated based on one or more of the clinical biomarker components. The extracted and classified segments of sensor data from the sensor(s) 102a-102e is used for input to the clinical biomarker estimation unit 107 for estimating one or more clinical biomarker(s) of the subject 102. Although each of the first set of ML model(s) may be described, by way of example only but is not limited to, operating on sensor data from a particular sensor or set of sensors, it is to be appreciated by the skilled person that each of the first set of ML model(s) may operate on sensor data from multiple sensor(s) for extracting relevant segments of sensor data from the multiple sensor(s) and classifying/estimating clinical biomarker components, biomarkers and the like therefrom for input to the second set of ML model(s) and/or as the application demands.

The clinical biomarker estimation unit 107 of the data processing unit 104 is configured for estimating one or more clinical biomarker(s) of the subject using a second set of ML model(s) configured to estimate the one or more clinical biomarker(s) of the subject based on the extracted and classified sensor data 106, which is input. The second set of ML model(s) are configured to construct an estimate one or more clinical biomarker(s) 108 of interest of the subject 102 based on the received extracted and classified portions of sensor data 106 and/or the corresponding one or more clinical biomarker components as input. The clinical biomarker estimation unit 107 may also include biomarker estimation units for estimating one or more biomarker(s) (or intermediate biomarkers) that may be used in the estimation or determination of the one or more clinical biomarker(s). Some of the second set of ML model(s) may be configured estimate a set of biomarker(s) (or intermediate biomarker(s)), in which further ML model(s) of the second set may be configured to construct an estimate of a clinical biomarker based on combining the set of biomarker(s) using a mathematical model or one or more ML model(s) of the second set of ML model(s) that are configured for estimating the clinical biomarker. The second set of ML model(s) may include one or more ML model(s), multiple ML model(s), one or more mathematical model(s), and/or multiple mathematical model(s) and the like, that are configured and/or coupled together for estimating one or more biomarkers, one or more clinical biomarker(s) and/or multiple clinical biomarker(s) of interest.

The sensor data 103a-103e from the sensor(s) 102a-102e associated with the subject 102 may be based on real-time sensor measurements of the subject 102. For example, the subject 102 may be performing specialist tests under direction of a clinician for determining one or more clinical biomarker(s) of interest. In another example, the sensor measurements 103a-103e of the subject may preferably be taken whilst the subject 102 performs their everyday or day-to-day activities. The sensor data 103a-103e may include sensor measurements of the subject 102 taken from a device configured for recording data during administration of a treatment to the subject 102. The device may be a wearable device and the like. In another example, the sensor data 103a-103e may include sensor measurements of the subject 102 taken continuously whilst the subject performs their everyday activities.

The sensor data 103a-103e from the sensors 102a-102e may be recorded for later upload and analysis by the clinical biomarker estimation system 100 when implemented on a local computing device, local server, remote computing device, remote server, cloud computing and/or distributed computing platform and the like. Alternatively, the sensor data may be analysed in-situ where the clinical biomarker estimation system 100 or portions thereof, such as sensor signal pre-processing unit 105 and/or clinical biomarker estimation unit 107, is implemented on a wearable apparatus by the subject 102. The sensor data may be continuously recorded and analysed by the clinical biomarker estimation system 100 as the subject performs everyday activities and/or clinical tests. For example, the sensor signal pre-processing unit 105 may be part of the wearable device for recording the extracted and classified segments of sensor data 106, which may be transmitted or sent to a remote computing device (e.g. local computing device, local server, remote computing device, remote server, cloud computing and/or distributed computing platform) for estimating clinical biomarker(s) 108 of the subject 102. Alternatively or additionally, the wearable device may also include a clinical biomarker processing unit 107 for processing the extracted and classified sensor data 106 and estimating clinical biomarker(s) of interest of the subject 102, which may then be sent or transmitted to a remote computing system or device for further processing or analysis by specialist personnel and the like.

FIG. 1c is a detailed diagram of a first example of the first and second processing stages 105 and 107 of the data processing pipeline of FIG. 1b for estimating clinical biomarker(s) 108a-108n from sensor data 103a-103e of one or more sensor(s) 102a-102e associated with the subject 102. In the first processing phase/stage 105, a first set of ML model(s)/mathematical model(s) 105a-105j receive the unstructured sensor data 103a-103e and are used to identify sensor signals of interest 112a-112d and 113a-113d from the unstructured sensor data 103a-103e. Identifying the relevant sensor signals of interest 112a-112d and 113a-113d that may be useful in estimating clinical biomarker(s) 108a-108n of the subject 102. This may include using the first set of ML model(s) 105a-105j to extract and/or classify the relevant portions of sensor data 112a-113c from the unstructured sensor data 103a-103e. The first set of ML model(S) 105a-105j are trained using one or more ML technique(s) to extract and/or classify the unstructured sensor data into the relevant sensor signals of interest 112a-112d and 113a-113d. This may be based on generating labelled training datasets based on identifying a plurality of test subjects bodily state(s) from sensor data during specialist clinician tests on the test subjects in relation to clinical biomarkers of interest.

The extracted portions of sensor data 112a-113c may be classified using one or more of the first set of model(s) to generate identified sensor signals of interest or extracted and classified segments of sensor data 106a to 106l. Each of the identified sensor signals of interest 106a-1061 include a plurality of portions of sensor data 112a-113c that correspond to the same classification or class. For example, identified signal of interest 106a (e.g. Identified Signal 1) includes the relevant sensor signals of interest 112a-112d that are classified as being associated with Identified Signal 1, identified signal of interest 1061 includes the relevant sensor signals of interest 113a-113c that are classified as being associated with Identified signal I. The identified sensor signals 106a-106 correspond to a biomarker sensor dataset that includes a set of biomarkers and corresponding relevant sensor signals of interest 112a-112d or 113a-113c that are functionally useful as clinical biomarker components for estimating one or more clinical biomarkers of interest 108a-108n. For example, Identified Signal 1 may correspond to a first clinical biomarker component and Identified signal I corresponds to a second clinical biomarker component that is identified from the sensor data 103. The identified sensor signals 106a-1061 are output from the first stage 105 as a biomarker sensor dataset, i.e. relevant portions/subcomponents or segments of extracted and classified sensor data from the unstructured sensor data 103.

The first stage 105 is configured for extracting and classifying portions of sensor data 112a-112d and 113a-113c from unstructured sensor data 103a-103e that are relevant for and/or useful for estimating clinical biomarkers 108a-108m of the subject 102. This includes receiving unstructured sensor data 103a-103e from one or more of a plurality of sensors 102a-102e associated with the subject 102. The unstructured sensor data 103a-103e may be processed by inputting the unstructured sensor data 103a-103e into one or more ML model(s) from a first set of ML model(s) configured to extract portions 112a-112d or 113a-113c of sensor data 103a-103e relevant for constructing and estimating clinical biomarker(s) 108a-108m of the subject 102.

For example, the unstructured sensor data 103a-103e that is output from each of the sensors 103a-103e may be processed by a different ML model of the first set of ML model(s), where each ML model is configured or trained to identify, extract and/or classify relevant portions of the corresponding sensor data. In another example, different ML model(s) of the first set of ML model(s) may process the same unstructured sensor data 103c from a particular sensor 102c in which each of the different ML model(s) extracts and classifies different portions of the same unstructured sensor data 103c. The extracted sensor data may include a first set of identified sensor signals of interest 112a-112d and a second set of identified sensor signals of interest 113a-113c. The first set of identified sensor signals of interest 112a-112d may be associated and classified with a first biomarker dataset 106a, which classifies or labels the first set of identified sensor signals of interest 112a-112d with one or more biomarker classes or labels representing the clinical biomarker components. The second set of identified sensor signals of interest 113a-113c may be associated and classified with a second biomarker dataset 1061, which classifies or labels the first set of identified sensor signals of interest 112a-112d with one or more biomarker classes or labels representing the clinical biomarker components. The first and second biomarker datasets 106a-1061 may form the extracted and classified segments of sensor data 106a-1061 for input to the second stage 107 in which a second set of ML model(s) 107 are configured to construct and estimate one or more clinical biomarker(s) of the subject 102.

In the second phase/stage of processing 107, the extracted and classified segments of sensor data 106a-1061 (e.g. identified sensor signals 106a-1061), which includes the portions or relevant sensor data 112a-112d or 113a-113c representing the clinical biomarker components (or identified signals of interest) that have been extracted from their original unstructured sensor data 103 are further processed using a second set of ML model(s)/mathematical models 107a-107m configured for outputting via output path 116 an estimate of one or more clinical biomarker(s) of interest 108a-108n. The extracted and classified segments of sensor data 106a-1061 are input path 114 to one or more of the second set of ML models, which may be configured to estimate one or more of the clinical biomarker(s) of interest 108a-108n.

FIG. 1d is a detailed diagram of a second example of the first and second processing stages 105 and 107 of the data processing pipeline 104 of FIG. 1c, in which second processing stage 107 is further modified to include a first subset of ML model(s) of the second set of ML model(s) for estimating biomarker(s) (or intermediate biomarkers) via feedback path 117 for use in estimating one or more of the clinical biomarker(s) of interest 108a-108n, a second subset of ML model(s) may be configured to estimate one or more of the clinical biomarker(s) of interest 108a-108n, and in which one or more ML model(s) of the second set of ML model(s) may be configured to receive as input at least one or more of: one or more estimated biomarker(s) via feedback path 117; estimated one or more clinical biomarker(s) of interest 108a-108n via feedback path 118; and one or more of the extracted and classified segments of sensor data 106a-1061 via input path 114.

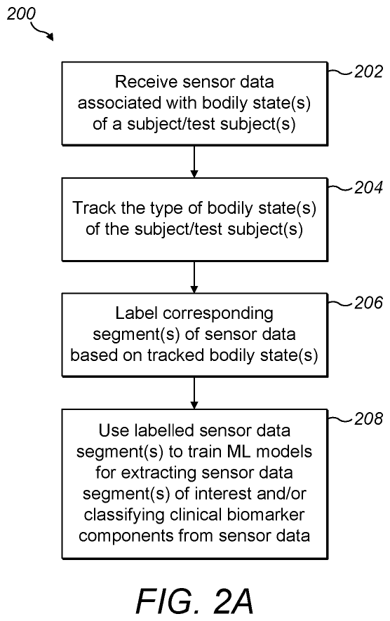

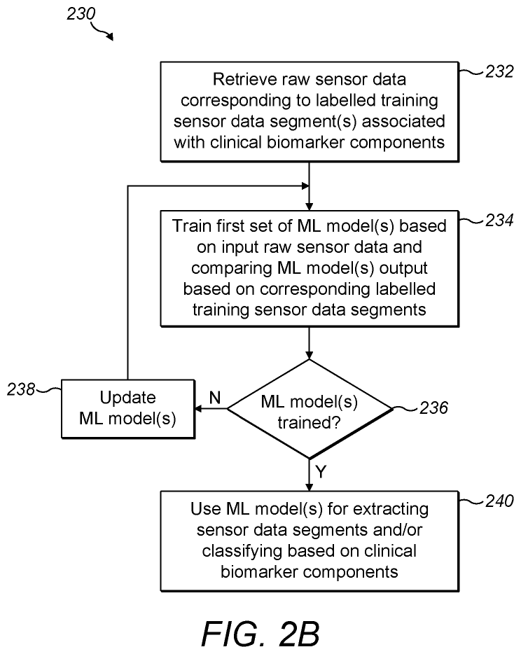

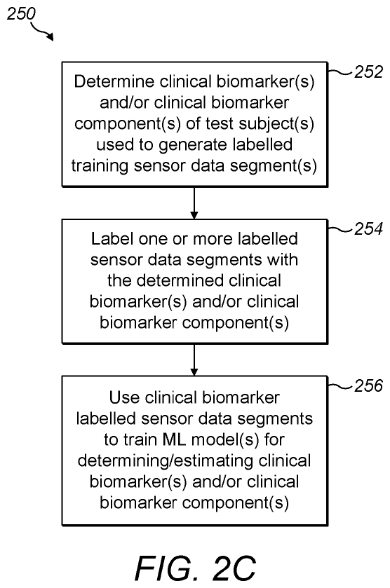

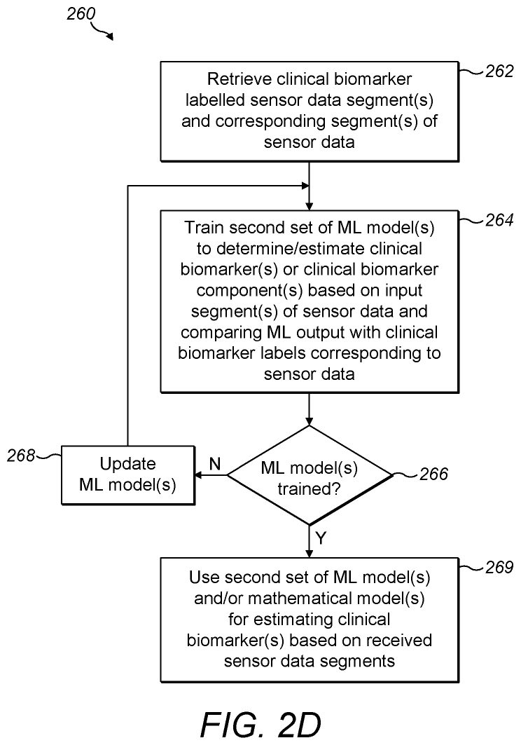

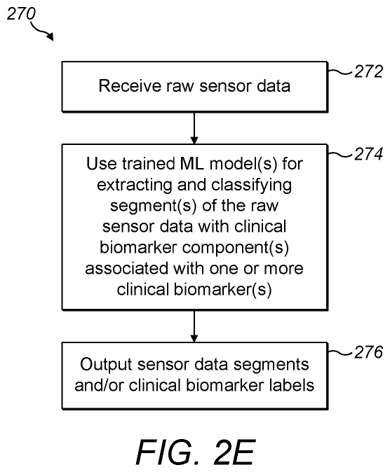

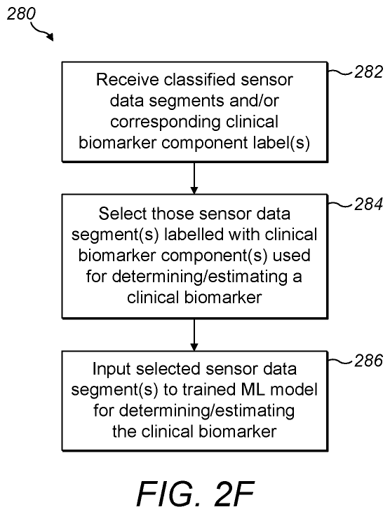

FIG. 2a is a flow diagram of a method 200 for generating labelled training dataset for use in the first processing stage 105 of the data processing pipeline 100 of FIG. 1b. The first processing stage 105 includes a first set of ML model(s) for extracting and/or classifying unstructured sensor data 103 into a suitable form for inputting to the second processing stage 107 that includes a second set of ML model(s) configured for estimating clinical biomarker(s) of interest 108. The first set of ML model(s) are required to be trained using one or more corresponding ML technique(s). Training the first set of ML model(s) for extracting portions of sensor data associated with estimating one or more clinical biomarker(s) of the subject may be based on a first labelled sensor training dataset associated with the one or more clinical biomarker(s). The first labelled sensor training dataset for a ML model may be generated based on a method or process 200 based on the following steps of:

In step 202, sensor data from sensors associated with a test subject in a bodily state associated with clinical biomarker(s) of interest is received. The bodily state of the test subject may be based on, by way of example only but is not limited to, movement of one or more body parts of the test subject in relation an activity the test subject is performing based on a specialised clinical test associated with one or more clinical biomarkers; operation of one or more bodily system, body parts and/or organs of the test subject based on a specialised clinical test associated with one or more clinical biomarkers; and the like. The raw sensor data may be stored for use in training the first set of one or more ML model(s).

In step 204, the one or more bodily state(s) of the test subject are tracked, where each different bodily state may defines a different type of clinical biomarker component for use in estimating a clinical biomarker of the subject. The different clinical biomarker components may be used for estimating one or more clinical biomarkers of the subject. For example, the bodily state may be based on activities or movements of the test subject, thus, step 204 may include tracking the type of activity the test subject performs in relation to a specialised test associated with one or more clinical biomarkers, where each type of activity defines a different clinical biomarker component for use in estimating a clinical biomarker.

In step 206, labelling, for each of the sensors associated with the test subject, each segment of sensor data of the test subject with a clinical biomarker component corresponding to the tracked bodily state that is determined for said each segment of sensor data. For example, this may include, where the bodily state may be based on activities or movements of the test subject, by way of example only but not limited to, labelling, for each of the sensors associated with the test subject, each segment of sensor data of the test subject with a clinical biomarker component corresponding to the tracked activity type determined for said each segment of sensor data.

In step 208, the labelled sensor training datasets may be stored for use in training one or more ML techniques to generate one or more ML model(s) configured to extract portions of sensor data of interest from received sensor data and/or classify each of the extracted portions of sensor data based on the clinical biomarker component(s). The clinical biomarker component(s) are based on biomarkers of the subject that have the functionality of being subcomponents of clinical biomarkers, which when combined or processed may be used for estimating one or more clinical biomarkers of interest.

FIG. 2b is a flow diagram of a method 230 for training a first set of machine learning (ML) technique(s) to generate a first set of ML model(s) for use in the first processing stage 105 of the data processing pipeline 100 of FIG. 1b. The training method 230 may be based on the stored sensor training dataset generated by method 200 of FIG. 2a. The labelled sensor training dataset includes a plurality of portions/segments or subcomponents of sensor data, each portion of sensor data associated with a label associated with estimating one or more clinical biomarker(s) or clinical biomarker component(s). The method 230 may include, by way of example only but is not limited to, one or more of the following steps of:

In step 232, the stored sensor data of one or more test subjects and the corresponding labelled sensor training data is retrieved. The labelled sensor training data may include one or more sensor data segments associated with the sensor data in which each sensor data segment is labelled with a corresponding clinical biomarker component. Multiple sets of sensor data and labelled sensor training datasets may be retrieved in which each labelled sensor training dataset and the corresponding raw sensor data may be associated with estimating the same or different ones of the one or more clinical biomarkers.

In step 234, one or more ML model(s) based on one or more ML technique(s) may be trained by inputting the retrieved raw sensor data to a first set of ML technique(s). The first set of ML technique(s) configured for training the first set of ML model(s) that output an indication of extracted sensor data segments and a classification of each segment in relation to one or more clinical biomarker component(s) associated with the one or more clinical biomarkers of interest. The output of each ML model may include an indication of the segments of sensor data that said each ML model has extracted and considers to be relevant and/or a classification of each extracted segment of the sensor data in relation to one or more of the clinical biomarker components. The output extracted and classified sensor data segments may be compared with the corresponding labelled sensor training data associated with the raw sensor data that is input to the ML model. This may be used to generate an error term or loss function associated with the ML technique for use in determining whether the ML model is trained. For example, if the error term or loss function reaches a certain predetermined error threshold, then the ML model may be considered to be trained.

In step 236, it is determined whether the or each ML model of the first set of ML models have been trained. For example, if the error term or loss function reaches a certain predetermined error threshold, then the ML model may be considered to be trained. If all the ML models have been trained, then the process 230 proceeds to step 240, otherwise the process 230 proceeds to step 238.

In step 238, one or more of the ML technique(s) that are considered not trained may be updated based on comparing the output indication of extracted sensor data segments and clinical biomarker component classification with the corresponding segments of the labelled sensor datasets. This may be used to generate an error term or loss function associated with the ML technique for use in updating the corresponding ML technique.

Steps 234 to 238 may be repeated until it is determined that the one or more ML technique(s) of the first set of ML models are validly trained.

In step 240, the ML technique(s) output the corresponding trained ML model(s), which are each configured for use in extracting sensor data segments and classifying each segment in relation to clinical biomarker components for use in estimating one or more clinical biomarkers.

FIG. 2c is a flow diagram of a method 250 for generating a further labelled training dataset for the second processing stage 107 based on the output of the first processing stage 105 of the data processing pipeline or system 100 of FIG. 1b. The second processing stage 107 includes a second set of ML model(s) for estimating one or more clinical biomarkers of interest based on extracted and/or classified unstructured sensor data 103 that has been processed by the first processing stage 105 into a suitable form for inputting to the second processing stage 107. The second set of ML model(s) are required to be trained using one or more corresponding ML technique(s). Training the second set of ML model(s) for estimating one or more clinical biomarker(s) of interest may be based on the first labelled training sensor dataset used for training the first set of ML model(s), which may be used as input to the second set of ML model(s) and on a second labelled training sensor dataset for use in comparing between the output of each ML model and the corresponding second labelled training sensor dataset. The second labelled sensor dataset may correspond to estimates or calculations of corresponding one or more clinical biomarkers in relation to the first labelled sensor dataset. For example, the second labelled sensor dataset(s) may include the first labelled sensor dataset(s) in which each of the first labelled sensor data(s) is further labelled with corresponding estimate(s) or calculations of clinical biomarker(s) calculated/determined in relation to the corresponding first labelled sensor dataset. Each second labelled sensor training dataset for an ML model may be generated using a method or process 250 based on the following steps of:

In step 252, a first labelled sensor dataset of a test subject (or one or more test subjects) used for training one or more of the ML models of the first set of ML model(s) is retrieved. If not already done so, then one or more clinical biomarker(s) of the test subject that are required to be estimated may be calculated based on the first labelled sensor dataset. Alternatively, when the first labelled sensor dataset of the test subject(s) was generated, the corresponding clinical biomarkers required to be estimated may have been calculated at the same time using conventional means. Furthermore, biomarkers, clinical biomarker components and/or other clinical biomarkers required to be used with one or more ML model(s) or mathematical model(s) of the second set of ML model(s) may also be calculated or generated at the time the first labelled sensor dataset is generated.

In step 254, labelling one or more segments of the first labelled sensor dataset of the test subject with at least one of: the corresponding calculated clinical biomarker(s); the corresponding calculated biomarkers; and the corresponding calculated clinical biomarker components; and the like.

In step 256, the labelled sensor dataset(s) are stored as the second labelled sensor dataset for use in training one or more ML techniques to generate one or more ML model(s) configured to estimate one or more corresponding clinical biomarker(s) of interest associated with the second labelled training dataset. The ML model(s) may then receive as input the output of the first set of ML model(s) including the extracted and classified segments of sensor data, each of which have been classified based on one or more clinical biomarker component(s).

FIG. 2d is a flow diagram of a method 260 for training a second set of ML technique(s) to generate a second set of ML model(s) for use in the second processing stage 107 of the data processing pipeline or system 100 of FIG. 1b. The training method 260 may be based on the stored second labelled sensor training dataset generated by method 250 of FIG. 2c. The second labelled sensor training dataset includes a plurality of portions/segments or subcomponents of sensor data in which one or more of the portions of sensor data are associated with a label or estimate of at least one of: a clinical biomarker; biomarker; and a clinical biomarker component. The method 250 may include, by way of example only but is not limited to, one or more of the following steps of:

In step 262, the stored first labelled sensor dataset and the corresponding second labelled sensor dataset of one or more test subjects generated using processes/method(s) 200 and 250 are retrieved. The first labelled sensor dataset is used as input to one or more ML technique(s) for training one or more ML models of the second set of ML models. The second labelled sensor dataset is used for training the ML techniques to output the correct or an estimate of the clinical biomarker associated with one or more segments of the sensor data of the first labelled sensor dataset.

In step 264, the first labelled sensor dataset is input to one or more of the second set of ML technique(s) for training one or more ML model(s) of the second set of ML model(s). The second set of ML model(s) are configured for estimating at least one of: one or more clinical biomarker(s); one or more biomarkers (or intermediate biomarkers); and one or more clinical biomarker component(s). The output of each ML model of the second set of ML models may include an estimate of: one or more clinical biomarker(s); one or more biomarker(s); one or more clinical biomarker component(s) and the like. The output estimates of one or more clinical biomarker(s); one or more biomarker(s); one or more clinical biomarker component(s) and the like may be compared with the corresponding second labelled sensor training dataset associated with the first labelled sensor training dataset. That is, for example, the output estimates of one or more clinical biomarkers based on the segments and labels of the first labelled sensor training data set are compared with the corresponding clinical biomarker labels of those segments. This comparison may be used to generate an error term or loss function associated with the ML technique for use in determining whether an ML model is trained. For example, if the error term or loss function reaches a certain predetermined error threshold, then the ML model may be considered to be trained.

In step 266, it is determined whether the or each ML model of the second set of ML models have been trained. For example, if the error term or loss function reaches a certain predetermined error threshold, then the ML model may be considered to be trained. If all the ML models have been trained, then the process 260 proceeds to step 270, otherwise the process 260 proceeds to step 268.

In step 268, one or more of the ML technique(s) that are considered not trained may be updated based on comparing the output estimates of one or more clinical biomarker(s); one or more biomarker(s); one or more clinical biomarker component(s) and the like with the corresponding estimates or calculations of the second labelled sensor training dataset. This may be used to generate an error term or loss function associated with the ML technique for use in updating the corresponding ML technique.

Steps 264 to 268 may be repeated until it is determined that the one or more ML technique(s) of the second set of ML models are validly trained. Alternatively or additionally, steps 264 to 268 may be repeated until it is determined that all one or more ML technique(s) of the second set of ML models are validly trained.

In step 269, the ML technique(s) that are used to train the ML model(s) are considered to be trained and so the corresponding data representative of the trained ML model(s) may be output. Each of the trained ML models may be configured for use with extracted and classified sensor data segments output from corresponding ones of the first set of ML model(s) for use in estimating one or more clinical biomarkers associated with the trained ML model.

Step 264 or 269 may further include using the output of one or more of the ML model(s) of the second set of ML models as an input to one or more other ML model(s) of the second set of ML models for estimating one or more clinical biomarker(s) of interest, in which the input to the one or more other ML model(s) may be based on at least one of: one or more of the estimated clinical biomarkers output by the one or more ML model(s); one or more of the biomarkers output by the one or more ML model(s); one or more of the clinical biomarker components output by the one or more ML model(s). Alternatively or additionally, steps 264 or 269 may further include combining of one or more of the estimated clinical biomarkers of one or more ML model(s) of the second set of ML model(s) by inputting at least one of: one or more estimated clinical biomarker(s) output by the one or more ML model(s); biomarker(s) output by the one or more ML model(s); clinical biomarker component(s) output by the one or more ML model(s); into a mathematical model for estimating at least one of: a further clinical biomarker of the subject; a further biomarker of the subject for input to another one or more ML model(s) or mathematical model(s) of the second set of ML model(s); a further clinical biomarker component of the subject for input to another one or more ML model(s) or mathematical model(s) of the second set of ML model(s). It is to be appreciated by the skilled person that the inputs and/or outputs of one or more ML model(s) of the second set of ML model(s) and/or one or more mathematical model(s), algorithm(s) or formulae and the like may be combined together for estimating one or more clinical biomarkers of a subject.

FIG. 2e is a flow diagram of a method 270 for using the first set of ML model(s) in the first processing stage of the data processing pipeline of FIG. 1b. The method 270 may include the following steps of: In step 272, raw sensor data from one or more sensor(s) associated with a subject may be received. In step 274, the received sensor data from one or more sensors of the subject may be processed by inputting the received sensor data to one or more corresponding ML model(s) of a first set of ML model(s), in which each of the one or more ML model(s) may be configured to extract and classify one or more portions/segments of sensor data corresponding to said each ML model that is relevant for constructing and estimating clinical biomarker(s) of interest of the subject. The one or more ML model(s) may be trained based on the method 230 in which ML technique(s) are trained to generate ML model(s) that are capable of outputting extracted and classified portions/segments of sensor data. The portions/segments of sensor data may be classified in relation to a biomarker dataset of one or more clinical biomarker component(s), where the one or more clinical biomarker component(s) may be used to estimate one or more clinical biomarkers of interest. In step 276, each of the ML model(s) of the first set of ML model(s) may output the extracted and classified portions of sensor data for input to one or more ML model(s) of a second set of ML model(s) configured to construct and estimate one or more clinical biomarker(s) of the subject.

FIG. 2f is a flow diagram of a method 280 for using the second set of ML model(s) in the second processing stage 107 of the data processing pipeline or system 100 of FIG. 1b. The method 280 may include the following steps for estimating one or more clinical biomarker(s) of a subject: In step 282, portions/segments of sensor data extracted and classified by one or more ML models of a first set of ML models associated with the one or more clinical biomarkers are received. The portions of sensor data may be extracted and classified output from the one or more ML model(s) of the first set of ML model(s) of the method 270 of FIG. 2g. These ML model(s) are configured for extracting the relevant portions of sensor data for constructing and estimating one or more clinical biomarkers of the subject. The extracted sensor data segments may be classified by the ML model(s) of the first set of ML model(s) in which each extracted sensor data segment is labelled with a clinical biomarker component from a set of biomarkers associated with one or more clinical biomarkers to be estimated. In step 284, those extracted sensor data segments that are labelled with one or more clinical biomarker components associated with a clinical biomarker to be estimated may be selected for input to one or more ML model(s) of the second set of ML model(s). In step 286, the selected segments of the sensor data and labels are input to the one or more ML model(s) of the second set of ML model(s) configured for estimating corresponding one or more clinical biomarkers of the subject.

Step 286 may further include using the output of one or more of the ML model(s) of the second set of ML models as an input to one or more other ML model(s) of the second set of ML models for estimating one or more clinical biomarker(s) of interest, in which the input to the one or more other ML model(s) may be based on at least one of: one or more of the estimated clinical biomarkers output by the one or more ML model(s); one or more of the biomarkers output by the one or more ML model(s); one or more of the clinical biomarker components output by the one or more ML model(s). Alternatively or additionally, step 286 may further include combining of one or more of the estimated clinical biomarkers of one or more ML model(s) of the second set of ML model(s) by inputting at least one of: one or more estimated clinical biomarker(s) output by the one or more ML model(s); biomarker(s) output by the one or more ML model(s); clinical biomarker component(s) output by the one or more ML model(s); into a mathematical model for estimating at least one of: a further clinical biomarker of the subject; a further biomarker of the subject for input to another one or more ML model(s) or mathematical model(s) of the second set of ML model(s); a further clinical biomarker component of the subject for input to another one or more ML model(s) or mathematical model(s) of the second set of ML model(s). It is to be appreciated by the skilled person that the inputs and/or outputs of one or more ML model(s) of the second set of ML model(s) and/or one or more mathematical model(s), algorithm(s) or formulae and the like may be combined together in any manner required for estimating one or more clinical biomarkers of a subject and/or as the application demands.

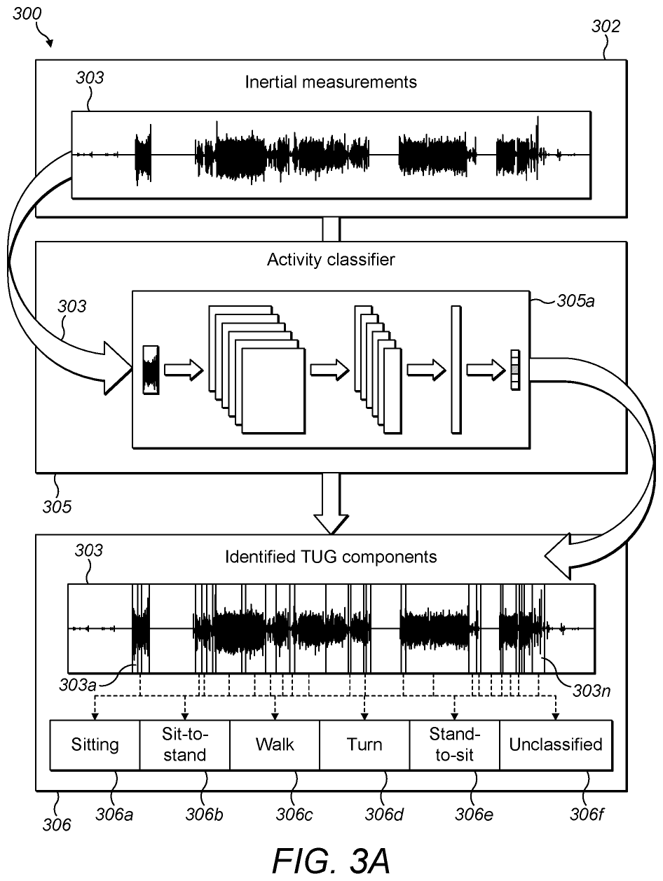

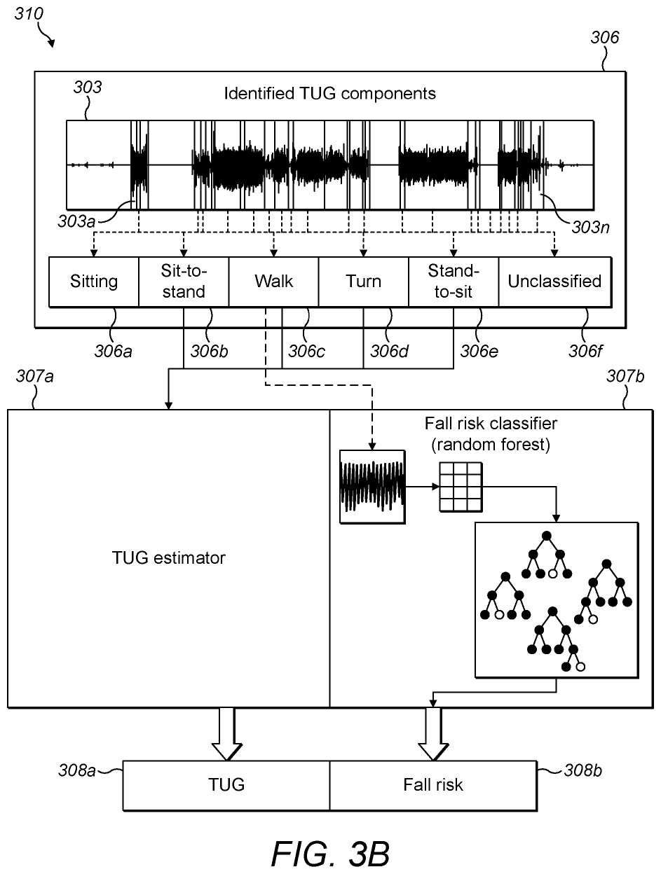

FIG. 3a is a schematic diagram of an example of a first ML model 305a for a first processing stage 300 of data processing pipeline/stage 104/105 or system 100 of FIGS. 1a-1d for estimating clinical biomarker(s) associated with timed up and go (e.g. a TUG clinical biomarker) or fall risk (e.g. a fall risk clinical biomarker). An inertial or mobility sensor 302 associated with a subject may output mobility sensor data 303 (e.g. inertial measurements from an inertial sensor associated with the subject) for input to a first set of ML model(s) 305 that includes a first ML model 305a for extracting and classifying relevant segments of the unstructured IMU data 303. This is an example of using the first ML model 305a to extract segments 303a-303n of the mobility sensor data 303 and classify the extracted segments 303a-303n based on several clinical biomarker components 306a-306f of the subject for use in estimating a mobility clinical biomarker (e.g. TUG clinical biomarker or fall risk clinical biomarker). The mobility sensor data 303 may be output from an inertial sensor 302 associated with the subject.

The mobility/inertial sensor 302 may be, by way of example only but is not limited to, an inertial measurement unit (e.g. IMU) that outputs inertial measurement data 303 generated based on bodily state(s) of the subject. For example, the subject may be performing a series of movements or daily activities that includes, by way of example only but is not limited to, at least sitting, walking, turning, sit-to-stand movements, stand-to-sit movements, running, falling or any other activity or movement. The bodily states based on movements performed by the subject are recorded as inertial measurements 303 by the IMU 302 associated with the subject. The inertial measurements 303 may be processed by the first ML model 305a for extracting and classifying segments 303a of the inertial measurements 303 of the IMU 302 that are relevant for use in estimating the clinical biomarker called Timed Up and Go (TUG).

In the standard clinical setting, TUG clinical biomarker is measured with a stopwatch as the time taken for a subject to perform the following test of: a) stand up from a chair, b) walk three meters, c) turn around, d) walk three meters to return to the same chair, and e) sit back down. Instead, the first and second stages of the data pipeline or system 104 may be configured and used to estimate or determined TUG time, which can instead be inferred from IMU sensor data 303 of the subject while the subject carries out their typical activities of daily living by extracting segments of IMU data 303 and classifying these segments based on clinical biomarker components associated with the TUG clinical biomarker.

The clinical biomarker components may be used to calculate an estimate of the TUG clinical biomarker from relevant extracted segments of the IMU data. This may be achieved by extracting the segments of IMU data and classifying each segment according to the clinical biomarker components of the TUG test. Estimating the TUG clinical biomarker may be achieved by calculating the time taken to carry out the above test, which may be derived from normal daily activities of the subject, selecting and stringing together a plurality of IMU measurement data segments 303a-303n classified by the required clinical biomarker components 306a-306f that are identified within the sensor data 303, then summing the times associated with these segments to estimate the TUG time.

In this example, the mobility or IMU sensor 302 is a 9-axis IMU containing an accelerometer, gyroscope and magnetometer that can be mounted on a subject's hip using a belt attachment. The IMU sensor 302 outputs IME measurement data associated with the accelerometer, gyroscope and magnetometer based on the subject's bodily state. The IMU measurement sensor data 303 may be normalised and passed through the first ML model 305a, which is an activity classifier 305a that is designed and trained to recognise the clinical biomarker components of the TUG test the subject's daily activity measured using the freely-moving IMU data including, by way of example only but not limited to: sit-to-stand, walking, turning, and stand-to-sit clinical biomarker components. The activity classifier 305a may be based on a convolutional neural network (CNN) ML technique that is trained as the encoder of a variational auto-encoder. The activity classifier 305a is trained to discard those IMU data segments that cannot be classified with adequately high certainty (e.g. ‘unclassified’), otherwise the activity classifier 305a is trained to identify IMU data segments 303a-303n, classify each identified IMU data segment 303a-303n based on the corresponding clinical biomarker components or labels thereof, and extract those identified IMU data segments 303a-303n that have been classified in relation to one of the clinical biomarker components with an adequate or suitably high certainty. The first ML model 305a outputs a TUG component dataset 306 which includes the extracted IMU data segments 303a-303n that are classified in relation to one or more of the clinical biomarker components 306a-306f (e.g. sit-to-stand, walking, turning, stand-to-sit, sitting, turning etc.)

FIG. 3b is a schematic diagram of an example of a second set of ML model(s) for the second processing stage 310 for use with data processing pipeline/stage 107 or system 100 of FIGS. 1a-1d for estimating clinical biomarker(s) associated with timed up and go (TUG) or fall risk of a subject. The first ML model 305a of FIG. 3a outputs a TUG component dataset 306 which includes the extracted IMU data segments 303a-303n that are classified in relation to one or more of the clinical biomarker components 306a-306f (e.g. sit-to-stand, walking, turning, stand-to-sit, sitting, turning etc.) The second processing stage 310 includes a second set of ML model(s) comprising a TUG estimator model 307a and a Fall Risk Classifier ML model 307b. For estimating the TUG clinical biomarker, the second processing stage 310 is configured to select from the TUG dataset 306 those IMU data segments classified with the TUG clinical biomarker component labels associated with the bodily states of “Sit-to-Stand”, “Walk”, “Turn” and “Stand-to-Sit” of the TUG test. The selected IMU data segments and corresponding TUG clinical biomarker component labels are input to the TUG estimator model 307a. The TUG estimator model 307a is configured to analyse the extracted IMU data segments that are labelled with the TUG clinical biomarker components. Estimating the TUG clinical biomarker of the subject may be achieved by calculating the time taken to carry out the above TUG test described with reference to FIG. 3a, which may be derived from normal daily activities of the subject by selecting and stringing together a plurality of IMU measurement data segments 303a-303n classified by the required clinical biomarker components 306a-306f that are identified within the sensor data 303 and then summing the times associated with these segments to estimate the TUG time of the subject.

For example, the IMU data segment(s) associated with Sit-to-Stand clinical biomarker component 306b may be used to measure the time the subject takes for Sit-to-Stand by the length of the IMU data segment(s). If there is more than one Sit-to-Stand IMU data segment, then these may be averaged to produce an average Sit-to-Stand time of the subject. The TUG estimator may also determine whether the subject has walked 3 meters by analysing the walking speed derived from IMU data segments associated with “Walk” clinical biomarker component 306c and assesses the time taken for the subject to walk 3 meters. The IMU data segment(s) associated with Turn clinical biomarker component 306d may be used to measure the time the subject takes for Turning by the length of the IMU data segment(s). If there is more than one Turn IMU data segment, then these may be averaged to produce an average Turn time of the subject. These IMU data segments may be analyses by the TUG estimator to determine the time the subject takes based on the IMU data segment(s) classified with the corresponding clinical biomarker component(s), the times of which may be summed to output an estimate of the TUG time clinical biomarker 308a of the subject.

The extracted IMU data segments 303a-303n that are classified in relation to one or more of the clinical biomarker components 306a-306f (e.g. sit-to-stand, walking, turning, stand-to-sit, sitting, turning etc.) is also used by the second processing stage 310 for estimating the Fall Risk of the subject using the Fall Risk Classifier ML model 307b. Following activity classification of the IMU measurement data described with reference to FIG. 3a, the IMU data segments 303a-303n that are selected and extracted for input to the Fall Risk Classifier ML model 307b are those with the required signal characteristics/clinical biomarker components for use in estimating fall risk. In this example, these include the IMU data segments 303a-303n that are classified/identified as “Walking” clinical biomarker components 306c. The Walking IMU data segments are used as inputs for to the Fall Risk Classifier ML model 307b of the second set of ML models. In this example, the Fall Risk Classifier ML model 307b is, by way of example only but is not limited to, a random forest classifier ML model. The random forest classifier ML model is trained based on the corresponding random forest ML technique, which is configured for estimating fall risk from one or more IMU data segments associated with the Walking clinical biomarker component. The random forest classifier model 307a is configured for identifying whether a subject (e.g. patients) has a high or low risk of falling according to their walking patterns, which are based on the “Walking” clinical biomarker components of the IMU data segments 303a-303n. The Fall Risk Classifier ML model 307b may output a Fall Risk score as the Fall Risk clinical biomarker 308b of the subject.

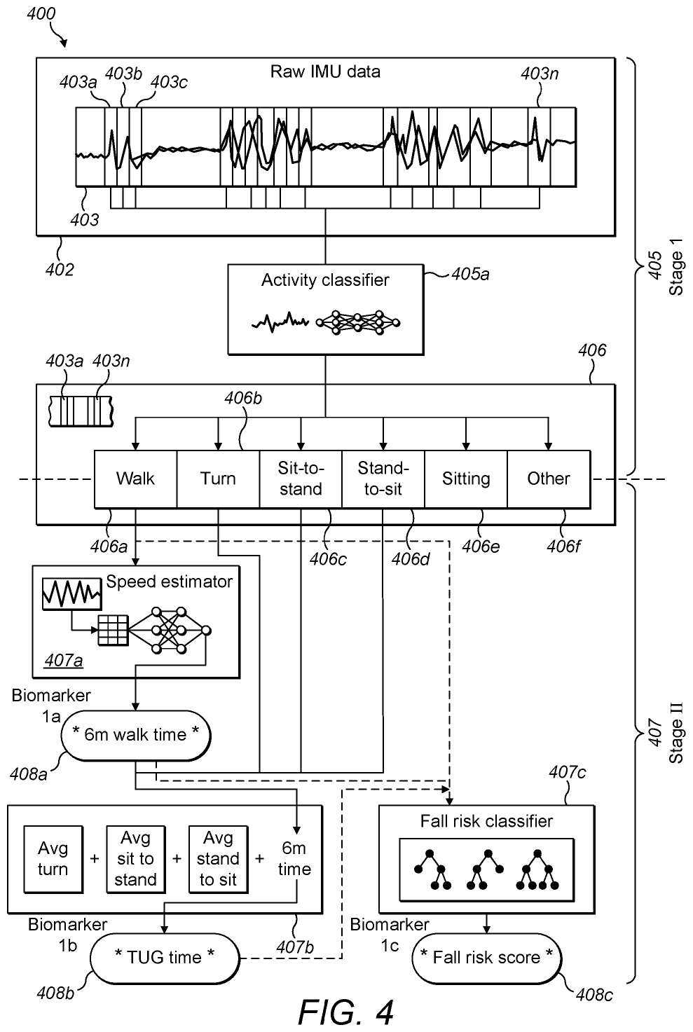

FIG. 4 is a schematic diagram of another example of a first set of ML model(s) 405a and second set of ML model(s) 407a-407c for use in the first processing stage 405 (e.g. STAGE I) and second processing stage 407 (e.g. STAGE II), respectively, of a data processing pipeline or system 400, and/or data processing pipeline/stages 105/107 or system 100 of FIGS. 1a-1d. The first and second processing stages may be used in the data processing pipeline 104, stages 105/107 or system 100 of any of FIGS. 1a-1d. Referring to FIG. 4, the ML model(s) 405a, 407a-407c are configured for estimating clinical biomarker(s) 408b and 408c (e.g. biomarkers 1b and 1c) associated with timed-up-and-go (TUG) and fall risk of a subject, respectively. In this example, an inertial or IMU unit 402 associated with a subject may output unstructured inertial measurement unit data 403 for input to the first ML model 405a.

Although TUG and fall risk clinical biomarkers are related to mobility of the subject, this is by way of example only and the invention is not so limited, it is to be appreciated by the skilled person that many other clinical biomarkers may be extracted from unstructured inertial measurement unit (IMU) data using this system or as the application demands. The IMU 402 may be part of an apparatus of device connected, fitted or strapped to the body of the subject, by way of example only but is not limited to, and is configured to output IMU data generated based on bodily state(s) of the subject. For example, the subject may be performing a series of movements or daily activities that includes, by way of example only but is not limited to, at least one or more of: sitting, walking, turning, sit-to-stand movements, stand-to-sit movements, running, falling or any other activity or movement. The bodily states based on movements performed by the subject are recorded or output as IMU data 403 from the IMU 402 associated with the subject.

As previously described with the data processing pipeline or system 100, the first processing stage 405 (e.g. STAGE I) is to use a first ML model 405a to extract IMU data segments of interest 403a-403n from the unstructured IMU data 403 and classify the extracted IMU data segments of interest 403a-403n in relation to one or more clinical biomarker components 406a-406f. The extracted IMU data segments of interest 403a-403n and corresponding clinical biomarker components 406a-406f form an IMU dataset 406. One or more or a plurality of the extracted IMU data segments of interest 403a-403n and corresponding clinical biomarker components 406a-406f from the IMU dataset 406 can be selected and applied or used for estimating one or more clinical biomarkers such as, by way of example only but not limited to, TUG clinical biomarker 408a and fall risk clinical biomarker 408b.

In this example, the first ML model 405a of the first set of ML model(s) is an activity classifier configured for identifying IMU data segments 403a-403n relevant to bodily states of the subject related to, by way of example only but not limited to, activity of the subject or any other bodily state derivable from unstructured IMU data 403. The activity of the subject may include activity classes or activity clinical biomarker components 406a-406f such as, by way of example only but not limited to, “walking” 406a, “turn”406b, “sit-to-stand” 406c, “stand-to-sit” 406d, “sitting” 406e and “other” 406f from unstructured IMU data 403. The segments that cannot be classified with adequate certainty, e.g. the IMU segments that are classified as “other” 406f, are discarded. The remaining IMU segments 403a-403n and corresponding clinical biomarker components 406a-406e are used for input to one or more second ML model(s) of the second set of ML model(s) configured for estimating one or more clinical biomarkers 408b and 408c and/or biomarkers 408a (e.g. biomarker 1a) and the like.

The first ML model 405a is an activity classifier generated from training an ML technique based on a neural network structure based on, by way of example only but not limited to, methods 200 and 210 with reference to FIGS. 2a-2b. For example, the first ML model 405 may be generated based on training an ML technique based on an variational autoencoder using Convolutional Neural Networks (CNNs), in which the trained encoder of the variational autoencoder is used for the first ML model 405a. The ML technique used to generate the first ML model 405a is based on a labelled training dataset derived from unstructured IMU data 403. For example, the IMU 402 may be configured to output 6 channels (e.g. 3 from triaxial accelerometer and 3 from triaxial gyroscope) of inertial IMU measurement data 403 that are collected from a hip-mounted IMU in a controlled environment while test subjects (e.g. participants) repeatedly carry out a series of activities that include the individual clinical biomarker components of the TUG test as described with reference to FIG. 3a. The individual clinical biomarker components of the TUG test may be the so-called TUG clinical biomarker components 406a-406f. While the test subjects carry out these activities, specialist personnel (e.g. an experimenter) may use a graphical user interface to track which activity associated with the set of TUG clinical biomarker components 406a-406f that a test subject of the plurality of test subjects is carrying out at a given time. The IMU data 403 can be segmented and labelled as it is collected based on the tracked and identified clinical biomarker components 406a-406f. The labelled IMU datasets may then be normalized and broken into shorter windows, and collected clinical biomarker component label are used as ground truth to train the first ML model to identify, recognise relevant IMU data segments 403a-403n from unstructured IMU data of a subject that corresponds to each TUG clinical biomarker component.

Once the first ML model 405a is trained as an activity classifier it is used for extracting and classifying relevant IMU data segments 403a-403n of unstructured IMU data 403 from an IMU 402 associated with a subject. The unstructured IMU data 403 may be based on, by way of example only but is not limited to, IMU data from 6 channels (e.g. 3 from triaxial accelerometer and 3 from triaxial gyroscope) of inertial measurements from a hip-mounted IMU on the subject (a remote user). In some examples of the invention, the subject is preferably carrying out unstructured and/or natural daily activities and the IMU data is being generated throughout the daily life of the subject. Alternatively or additionally, the subject may be carrying out a specific TUG test or set of structured activities/movements in a clinic under supervision of specialist personnel (e.g. a clinician) in which the IMU data is generated during the TUG test. In any event, the unstructured IMU data 403 output from the IMU 402 is input to the first ML model 405a, which is configured for processing IMU data 403 and outputting an IMU dataset 406 comprising identified IMU segments 403a-403n and corresponding clinical biomarker components 406a-406e (or activity classes) corresponding to TUG clinical biomarker components 406a-406e. IMU data segments that are classed as ‘unclassified’ or “other” because they do not correspond to a TUG component with a certainty above a predefined threshold may be labelled as such and/or discarded. The IMU dataset 406 is used for input to one or more second ML model(s)/mathematical model(s) of the second set of ML model(s) configured for estimating one or more biomarkers 408a and/or one or more clinical biomarkers 408b and 408c (e.g. TUG clinical biomarker 408b and fall risk clinical biomarker 408c) and the like.

Following the first processing stage 405 (e.g. STAGE I), different types or segments of the IMU dataset 406 can be selected and/or extracted and separately analysed by passing the selected IMU data segments 403a-403n and corresponding clinical biomarker(s) through further processing steps comprising a second set of ML model(s) and/or mathematical methods 407a-407c for determining and estimating at least one of: one or more biomarker(s); one or more other biomarker(s); one or more clinical biomarker(s) and/or clinical biomarker component(s) and the like. For some of the estimated biomarkers, a previously-calculated or estimated biomarker may be used as input to a further processing step comprising a ML model or mathematical model of the second set of ML model(s)/mathematical model(s) 407a-407c. The type of processing using the second set of ML model(s) are different for different biomarkers, where some require additional complex ML technique(s) for generating ML models 407a and 407c in relation to biomarkers 408a and 408c, respectively, whilst other biomarker(s) may require simpler processing of the extracted IMU data segment(s) 403a-403n and corresponding clinical biomarker components 406a-406f of various bodily states of the subject. For example, calculation of the frequency and length of certain activity classes (e.g. clinical biomarker components of the subject) of the subject. For example, active time can be calculated as the total length of all segments that are classified as non-resting, or average time for sit-to-stand transition is the average length of labelled sit-to-stand segments. One or more of the biomarker(s) may be combined or input to further processing of the second processing stage 407 (e.g. STAGE II) for estimating and outputting clinical biomarker(s) 408a and 408b (e.g. TUG clinical biomarker and Fall Risk clinical biomarker).

The second processing stage 407 (e.g. STAGE II) includes a second set of ML model(s)/mathematical model(s) 407a-407c in which one or more biomarker(s), one or more clinical biomarkers are estimated or calculated based on the IMU data 406 output from the first set of ML model(s). For example, the output of one or more of the ML model(s) of the second set of ML models configured for estimating one or more biomarkers or clinical biomarkers may be used as an input to one or more other ML model(s) of the second set of ML models for estimating one or more clinical biomarker(s) of interest. The input to the one or more other ML model(s) may be based on at least one of: one or more of the estimated clinical biomarkers output by the one or more ML model(s) of the second set of ML model(s); one or more of the biomarkers output by the one or more ML model(s) of the second set of ML model(s); one or more of the clinical biomarker components output by the one or more ML model(s) of the first set of ML model(s); and any other biomarker or clinical biomarker and the like output from one or more mathematical model(s) of the second set of ML model(s). It is to be appreciated by the skilled person that the outputs of the one or more ML model(s) 405a of the first set of ML models, and the inputs and/or outputs of one or more ML model(s) 407a-407c of the second set of ML model(s) and/or one or more mathematical model(s), algorithm(s) or formulae and the like may be combined together for estimating one or more clinical biomarkers of a subject.

In the example data processing pipeline or system 400, the second stage of processing 407 (e.g. STAGE II) includes a plurality of ML models 407a-407c of a second set of ML models/mathematical models 407a-407c. A first ML model 407a of the second set of ML model(s) receives selected IMU data segments 403a-403n from the IMU dataset 406 that correspond to those type(s) of clinical biomarker component(s) associated with speed of the subject. In this example, the IMU data segments 403a-403n are those IMU data segments 403a-403n corresponding to the clinical biomarker component of “Walking” 406a. The ML model 407a of the second set of ML models outputs an estimate of an biomarker, the so-called 6 m walk time biomarker 408a. A second ML model 407b of the second set of ML model(s) may be based on a mathematical model for calculating TUG time clinical biomarker 408b, the second ML model 407b receives the following input data based on: those IMU data segments 403a-403n of the IMU dataset 406 corresponding to the clinical biomarker components of “Turn” 406b, “Sit-to-Stand” 406c, “Stand-to-Sit”; and the biomarker of “6 m walk time” estimated from the first ML model 407a of the second set of ML model(s). Based on these inputs, the second ML model 407b is configured to estimate the TUG time clinical biomarker 408a of the subject. Finally, a third ML model 407c of the second set of ML models is configured to estimate fall risk clinical biomarker and receives the following input data based on: those IMU data segments 403a-403n of the IMU dataset 406 corresponding to the clinical biomarker component of “Walking” 406a; the biomarker of “6 m walk time” estimated from the first ML model 407a of the second set of ML model(s); and the TUG clinical biomarker 408a estimated from the second ML model 407b. Based on these inputs, the third ML model 407c of the second set of ML models is configured to estimate a fall risk score, which is output as the fall risk clinical biomarker 408b of the subject. Thus, the second stage of processing 407 (e.g. STAGE II) uses several ML models/mathematical models of the second set of ML models for estimating one or more clinical biomarker(s) of the subject, which include, by way of example only but is not limited to, TUG clinical biomarker 408a of the subject and fall risk clinical biomarker 408b of the subject.

In this example, the first ML model 407a of the second set of ML models is a walking speed estimator that is configured to, when given segments of IMU walking data from the IMU dataset 406, estimates the distance travelled during that walking period and therefore infers an biomarker 408a based on the average walking speed of the subject. The first ML model 407a is based on a physics-based model of human position based on IMU measurement data and refined using a neural network. The neural network may be trained using a labelled training dataset from IMU measurements of a plurality of test subjects. For example, labelled training datasets may be generated using IMU data from 9 channels (3 from triaxial accelerometer, 3 from triaxial gyroscope and 3 from triaxial magnetometer) of an IMU 402 associated with the subject for generating IMU measurements associated with the bodily state of the subject. The IMU may be part of an apparatus fitted or strapped to the subject for measuring the activity of the subject. For example, IMU data may be collected from a hip-mounted IMU unit over, by way of example, a wireless connection (e.g. Wi-Fi) in a gait laboratory so that test subjects position and movements can be tracked continuously during walking and aligned with collected IMU data. The change in the position of a test subject during moving time intervals or windows (e.g. two-second windows) may be used to label segments of normalised IMU data with participant walking speed. The first ML model includes a physics-mathematical model to estimate speed and a neural network model to refine the speed estimate. For example, IMU data segments associated with walking are processed through the physics-based mathematical model to estimate speed, the output of which is compared to the true labels and a neural network applied to the IMU data and the intermediate outputs of the physics-based model is used to improve the outputs and estimate walking speed of the subject. Thus, the first ML model of the second set of ML models is configured to estimate walking speed of the subject based on input IMU data segments 403a-403n associated with the clinical biomarker component of “Walking”. These IMU data segments may comprise IMU measurements from 9 channels of normalised and windowed IMU data segments 403a-403n that have been labelled as ‘walking’ segments by the first set of ML model(s) configured as a activity classifier 405a. The first ML model 407a of the second set of ML models processes the relevant IMU data segments corresponding to the “walking” clinical biomarker component and is configured to output data representative of walking speed of the subject for each segment/window (e.g. speed may be in m/s or any other unit), which may then be averaged, converted and/or calculated to estimate a first biomarker 408a associated with the TUG test that indicates, by way of example only but is not limited to, the average time that would be taken for a given user to walk 6 metres (e.g. an “6 m walk time” biomarker). Although the first biomarker 408a is based on, by way of example only and not limited to, an estimate of the 6-metre walk time of a subject, it is to be appreciated by the skilled person that the example of FIG. 4 is to illustrate how different biomarkers may be added to the system 400 or 100 and that any one or more other biomarkers or clinical biomarkers may be estimated by one or more ML model(s) or mathematical model(s) of the second set of ML model(s), which can be combined and/or used for estimating one or more clinical biomarkers of the subject as the application demands.

The second ML model 407b of the second set of ML model(s) may be based on one or more mathematical model(s) for calculating or estimating the TUG time clinical biomarker 408b of the subject. The second ML model 407b receives the following input data based on: those IMU data segments 403a-403n of the IMU dataset 406 (e.g. from an IMU sensor, which may be a 9-axis IMU containing an accelerometer, gyroscope and magnetometer that is mounted on a user's hip using a belt attachment) corresponding to the clinical biomarker components of “Turn” 406b, “Sit-to-Stand” 406c, “Stand-to-Sit”; and the first biomarker of “6 m walk time” 408a (e.g. a “6 m walk time” biomarker) estimated from the first ML model 407a of the second set of ML model(s). Based on these inputs, the second ML model 407b is configured to estimate the TUG time clinical biomarker 408a of the subject. Biomarker 1b. Timed Up and Go. The second ML model 407b estimates the TUG clinical biomarker by analysing the lengths of each type of extracted IMU data segments 403a-403n) corresponding to the clinical biomarker components of “Turn” 406b, “Sit-to-Stand” 406c, “Stand-to-Sit” 406d to estimate “Turn”, “Sit-to-Stand”, “Stand-to-Sit” TUG components, which are combined together along with the first biomarker 408a estimate of the 6-metre walk time by, for example, summing these TUG components and first biomarker 408a for outputting an estimate of the TUG time clinical biomarker 408b of the subject.

Typically fall risk is conventionally assessed for elderly patients through in-person screening using questionnaires that reflect general mobility. The third ML model 407c of the second set of ML models is configured to estimate fall risk clinical biomarker based on the following quantitative input data of: those IMU data segments 403a-403n of the IMU dataset 406 corresponding to the clinical biomarker component of “Walking” 406a; the first biomarker 408a or “6 m walk time” biomarker estimated from the first ML model 407a of the second set of ML model(s); and the TUG clinical biomarker 408b estimated from the second ML model 407b of the second set of ML model(s). Based on these inputs, the third ML model 407c of the second set of ML models is configured to estimate a fall risk score, which is output as the fall risk clinical biomarker 408b of the subject. Biomarker 1c. Fall Risk

The third ML model 407c for estimating fall risk is generated based on, by way of example only but is not limited to, an ML technique based on a random forest structure. The third ML model 407c uses the same IMU dataset 406 as used by the first and second ML models 407a and 407b of the second set of ML model(s). Following activity classification and generation of the IMU dataset 406 by the first set of ML model(s), signal characteristics are extracted from IMU data segments 403a-403n that are identified as corresponding to the clinical biomarker component “Walking” (e.g. walking segments) and used as inputs for to the ML technique that generates the third ML model 407c of the second set of models in the form of a random forest classifier configured for identifying subjects as having a high or low risk of falling according to their walking patterns from IMU data segments classified as “Walking” and/or also, by way of example only but not limited to, the TUG time clinical biomarker 408b and first biomarker 408a of the subject.

The ML technique based on a random forest structure is trained for generating the third ML model 407a as a random forest classifier for estimating fall risk scores or clinical biomarkers of a subject. The ML technique for generating the random forest classifier may use a labelled training dataset that includes data representative of the 6-metre walk time of test subjects, TUG time of test subjects and walking features extracted from IMU data segments classified as “Walking” of the test subjects (e.g. 6/9 channels of inertial measurements are collected from a hip-mounted IMU while test subjects walk around a controlled environment). Test subjects are also given a clinically-validated questionnaire which is also used to assess their fall risk. IMU data segments of walking are broken into time intervals or windows (e.g. two-second windows) and turned into features generated from basic statistics and signal processing including, by way of example only but not limited to, means, standard deviations and frequency domain characteristics of each channel of the IMU data segments associated with walking. These sets of features, each corresponding to a walking segment, are labelled with the test subject's risk scores and are used as a labelled training dataset for input to the ML technique for generating the random forest classifier for estimating fall risk of a subject. The trained random forest classifier is used as the third ML model 407c of the second set of model(s) of the second processing stage 407 (e.g. STAGE II).

In this example, a subject is provided with an apparatus including an IMU 402 for outputting IMU measurements 403 of the subject (e.g. 6/9 channel inertial measurements collected from a hip-mounted IMU associated with the subject). The third ML model 407c receives input data based on: the 6-metre walk time of the subject output from the first ML model 407a of the second set of ML model(s), TUG time of test subjects output from the second ML model 407b of the second set of model(s) and walking features extracted from IMU data segments classified as “Walking” of the subject by the first ML model 405a of the first set of ML model(s). The third ML model 407c of the second set of ML model(s) processes the input data and outputs a fall risk score clinical biomarker 408c of the subject.

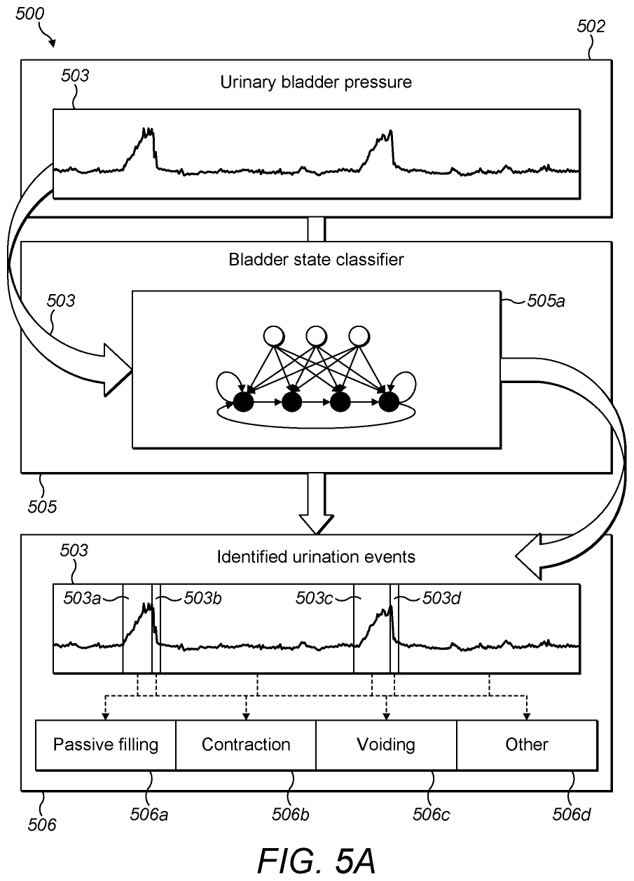

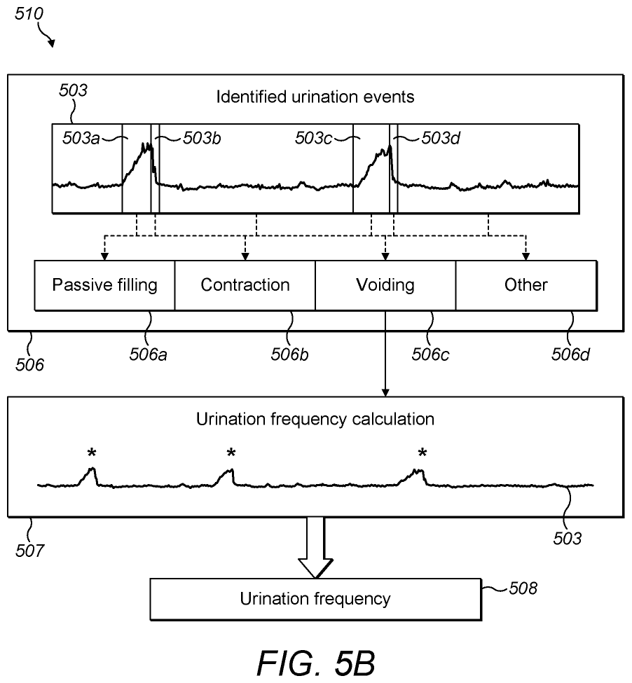

FIG. 5a is a schematic diagram of an example of a first ML model 505a of the set of ML model(s) for use in the first processing stage 500/505 of data processing pipeline/stage 104/105 or system 100 of any of FIGS. 1a-1d for estimating clinical biomarker(s) associated with urination events of a subject. In this example, a clinical biomarker based on frequency of urination of a subject may be determined from unstructured pressure sensor data 503 of a pressure sensor 502 implanted in the bladder of a subject. The pressure sensor 502 associated with the subject is used to monitor urination events of the subject. This is particularly useful clinical biomarker for subjects or patients who experience urinary incontinence. This is an example of using the first ML model 505a configured to extract segments 503a-503d of the unstructured pressure sensor data 503 output from the pressure sensor 502 associated with the subject. The first ML model 505a is also configured to classify the extracted segments 503a-503n based on several clinical biomarker components 506a-506d of the subject that are related to or for use in estimating a clinical biomarkers associated with urination events of the subject. The clinical biomarker components 506a-506d may include, by way of example only but is not limited to, a passive filing event, contraction event, voiding event, and other events. The unstructured pressure sensor data 503 of the subject is output from the pressure sensor 502 associated with the subject for input to the first ML model 505a. The first ML model 505a is configured to output a pressure sensor dataset 506 that includes, by way of example only but is not limited to, the relevant extracted portions/segments of pressure data 503a-503d in which each pressure data segment 503a-503d is labelled/classified based on the clinical biomarker components 506a-506d.

In this case, the first ML model 505a is configured as a bladder state classifier for identifying voiding events from the pressure sensor data 503. The bladder state classifier is based on training a ML technique for generating hidden Markov models. The hidden Markov model differentiates between typical passive filling 506a, contraction 506b, voiding 506c, and other changes in pressure 506d, such as due to environmental pressure. The first ML model 505a is derived from training the ML technique based on a labelled training dataset, which may be based on the methods 200 and 210 as described with reference to FIGS. 2a and 2b. For example, a labelled training dataset may be generated from unstructured pressure sensor data collected from, by way of example only but not limited to, bladder pressure measurements from an implanted bladder pressure sensor associated with one or more test subject(s) in a controlled environment. Specialist personnel or experts may manually label the phases of bladder voiding in the unstructured pressure sensor data. These labels are used on time intervals, segments or windows of the pressure sensor data that have been normalised, smoothed and converted to simple features (e.g. using mean, standard deviation etc.) to train the ML HMM technique to recognise phases from bladder pressure sensor measurements. Once the ML HMM technique is trained, a HMM model is output as the first ML model 505a (e.g. Bladder State Classifier) that is capable or configured to identify, extract segments of the unstructured pressure sensor data and classify phases or related clinical biomarker components 506a-506d in relation to each identified/extracted segment of the unstructured pressure sensor data. Thus, the first ML model 505a of the first set of ML model(s) is configured to receive input data based on, by way of example only but not limited to, unstructured pressure sensor data associated with features extracted from 1 channel of normalised and windowed bladder pressure measurements and configured to output clinical biomarker components associated with bladder phases passive filling 506a, contraction 506b, voiding 506c and/or other 506d for undefined pressure data windows if a predefined classification threshold of certainty is not met.

Although the first ML model 505a may output extracted segments of pressure sensor data 503a-503d and corresponding clinical biomarker components 506a-506d, it is to be appreciated by the skilled person that the first ML model 505a may only output the identified phases/classes of the clinical biomarker components 506a-506d for each segment of the pressure sensor data 503a-503d depending on the requirements and complexity of the second set of ML model(s) and clinical biomarkers that are to be estimated. Sometimes, the second set of ML model(s) may require both the pressure sensor dataset 506 including both the extracted segments of the pressure sensor measurements 503a-503d and corresponding labels of the clinical biomarker components 506a-506d, but other clinical biomarkers may require only an indication of each of the phases or clinical biomarker components 506a-506d in relation to the segments of pressure sensor measurements 503a-503d. For example, the urination frequency clinical biomarker, which is an indication of the frequency of voiding events, may only require, by way of example only but is not limited to, the clinical biomarker component portion of the pressure sensor dataset 506 related to voiding events 506c.

FIG. 5b is a schematic diagram of an example of a second set of ML/mathematical model(s) 507 for the second processing stage 510 for use with data processing pipeline/stage 107 of FIGS. 1a-1d for estimating clinical biomarker(s) associated with voiding urination events of a subject. In this example, the second set of ML model(s) 507 comprises a urination frequency calculation mathematical model 507. The first ML model 505a of FIG. 5a outputs at least the clinical biomarker component portion of the pressure sensor dataset 506 related to voiding events 506c for input to the urination frequency calculation mathematical model 507 of the second set of ML model(s)/mathematical model(s). The urination frequency calculation mathematical model 507 is configured to receive the voiding events 506c output from the pressure sensor dataset 506 of the first ML model 505a of FIG. 5a. The urination frequency calculation mathematical model 507 is further configured to determine the frequency with which voiding events 506c of the subject occurs, and outputs a urination frequency clinical biomarker 508 that can be calculated/estimated over the course of the day of the subject.

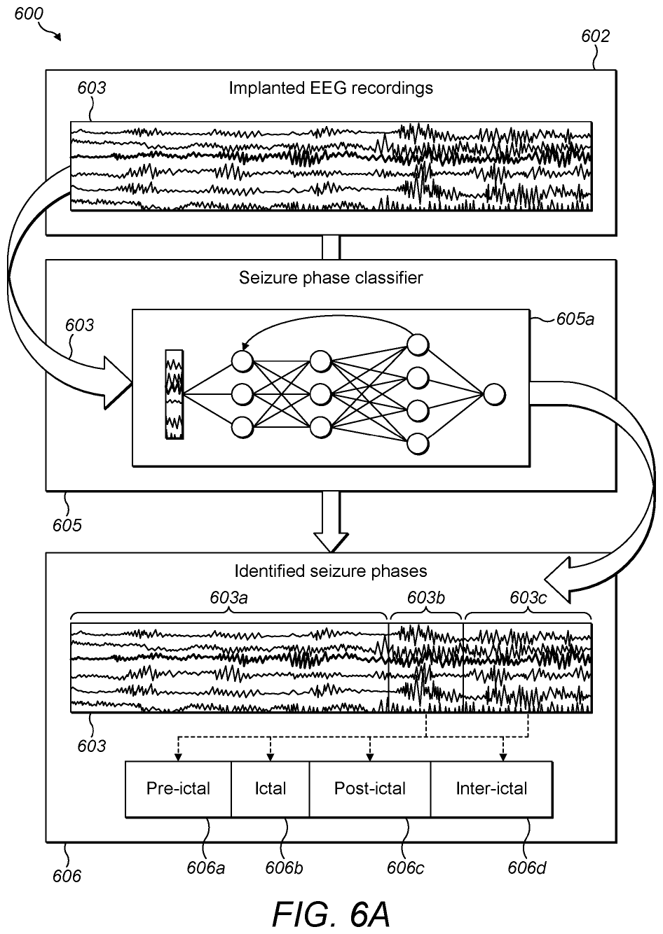

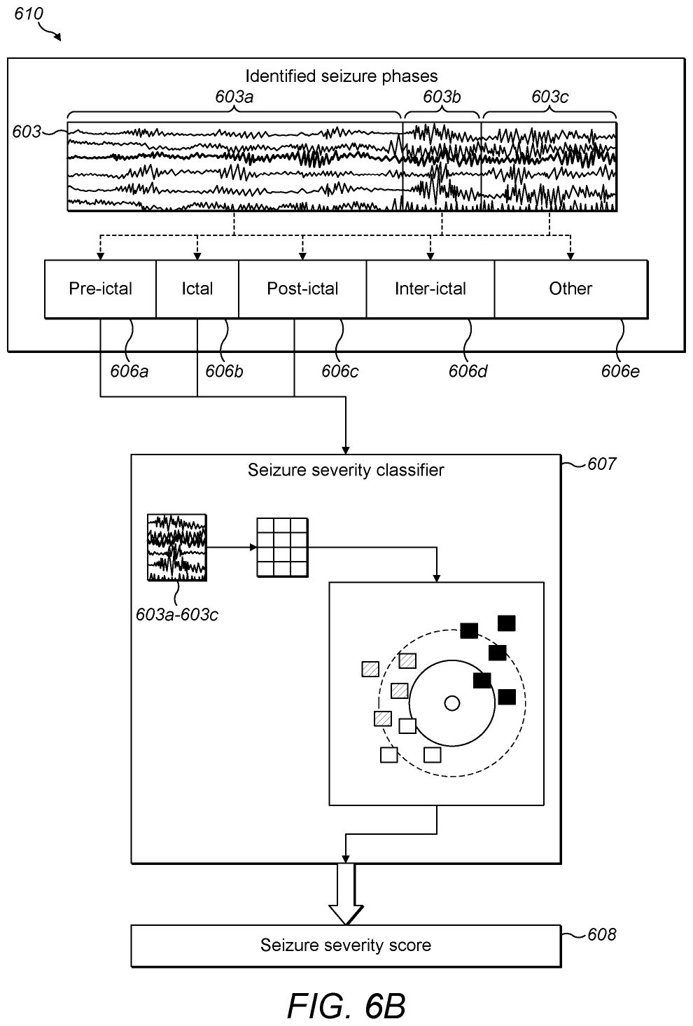

FIG. 6a is a schematic diagram of an example of another first ML model 605a for a first processing stage 600/605 of data processing pipeline/stage 104/105 or system 100 of FIGS. 1a-1d for estimating clinical biomarker(s) associated with seizure events of a subject. One or more electroencephalography (EEG) electrode(s)/sensor(s) 602 associated with a subject may output EEG trace data 603 for input to the first set of ML model(s) 605 that includes the first ML model 605a for extracting and classifying relevant segments of the unstructured EEG trace data 603. The one or more EEG electrodes/sensors 602 may be implanted on the cortex of the subject (e.g. patients) and collected to detect the number and duration of seizures experienced by an epileptic patient during a period of time, by way of example only but not limited to, a day, several days or a week or any period of time or time interval and the like.

This is an example of using the first ML model 605a to extract segments 603a-603c of the EEG trace data 603 and classify the extracted segments 603a-603c based on several clinical biomarker components 606a-606f of the subject associated with EEG readings and relevant for use in estimating a seizure event clinical biomarkers of the subject. The EEG trace data 603 may be output from an the EEG electrodes/sensor(s) 602 associated with the subject for input to the first ML model 605a, which outputs an EEG trace dataset 606 that includes extracted EEG trace segments 603a-603c classified based on several clinical biomarker components 606a-606f. Conventionally, a specialist must manually find and analyse epileptic periods in EEG traces of a subject. Instead, the first ML model 605a of the data processing pipeline us configured to classify windows/segments 603-603c of the raw or unstructured EEG trace data 603 based on clinical biomarker components such as, by way of example only but is not limited to, pre-ictal 606a, ictal 606b, post-ictal 606c, and inter-ictal 606d signals or other signals 606e.

The first ML model 605a is configured to be a seizure phase classifier and is generated by training a ML technique based on a neural network structure. In this example, the neural network structure is based on a Long-Short-Term-Memory (LSTM) recurrent neural network, which is trained to identify segments of EEG trace data 630a-630c and classify the identified segments of EEG trace data 630a-630c based on clinical biomarker component labels such as, by way of example only but is not limited to, pre-ictal 606a, ictal 606b, post-ictal 606c, and inter-ictal 606d signals or other signals 606e. The seizure phase classifier of the first ML model 605a may be trained to discard those EEG trace data segments that cannot be classified with adequately high certainty (e.g. ‘other’), otherwise the seizure phase classifier of the first ML model 605a is trained to identify EEG trace data segments 603a-603c, classify each identified EEG trace data segment 603a-603n based on the corresponding clinical biomarker components or labels thereof, and extract those identified EEG trace data segments 603a-603n that have been classified in relation to the one of the clinical biomarker components 606a-606d with an adequate or suitably high certainty.

The ML technique based on a Long-Short-Term-Memory (LSTM) recurrent neural network structure is trained for generating the first ML model 605a as a seizure phase classifier for extracting and classifying segments of EEG trace data 603a-603c based on the clinical biomarker components such as, by way of example only but is not limited to, pre-ictal 606a, ictal 606b, post-ictal 606c, and inter-ictal 606d signals or other signals 606e. The ML technique for generating the seizure phase classifier may use a labelled training dataset that is derived from unstructured EEG trace data 603 of a plurality of test subjects. For example, the EEG electrodes/sensors 602 may be configured to output, by way of example only but not limited to, N channels of EEG measurements (e.g. 16 channels of EEG measurements) that are recorded from subdural EEG electrodes/sensors 602 that have been implanted on the cortex of the test subjects with epilepsy and collected in a controlled environment. Specialist experts manually label the seizure phases based on professional understanding of the EEG traces and observed events. The seizure phase labelling may be based on, by way of example only but not limited to, clinical biomarker components such as, by way of example only but is not limited to, pre-ictal 606a, ictal 606b, post-ictal 606c, and inter-ictal 606d signals or other signals 606e. The EEG trace data 603 is segmented and labelled as it is collected. The labelled and segmented EEG trace data is windowed and the expert labels of clinical biomarker components can be used to train the LSTM RNN ML technique to recognise seizure phases compared to normal brain activity. The trained LSTM RNN ML technique is output as the first ML model 605a for extracting and/or classifying the EEG trace data 603 of a subject in relation to the clinical biomarker components 606a-606e. The first ML model 605a is then configured to receive EEG trace data 603 and process based on the seizure phase classifier LSTM RNN, which outputs an EEG trace dataset 606 that includes the EEG trace data segments 603a-603c in which each EEG trace data segment is classified by a corresponding clinical biomarker components 606a-606e, which define seizure phases or undefined if predefined threshold of certainty is not met.

FIG. 6b is a schematic diagram of an example of a second set of ML model(s) 607 for the second processing stage 610 for use with data processing pipeline/stage 107 or system 100 of FIGS. 1a-1d for estimating clinical biomarker(s) associated with seizure events of a subject. The first ML model 605a of FIG. 6a outputs a EEG dataset 606 which includes the EEG trace data segments 603a-603c in which each EEG trace data segment is classified by a corresponding clinical biomarker components 606a-606e (e.g. pre-ictal 606a, ictal 606b, post-ictal 606c, and inter-ictal 606d signals or other signals 606e etc.) In this example, the second processing stage 610 includes second ML model 607 comprising a seizure severity classifier configured for estimating a seizure severity score clinical biomarker 608. In this example, the second processing stage 610 is configured to select from the EEG dataset 606 those EEG trace data segments classified with the clinical biomarker component labels associated with the bodily states of, by way of example only but is not limited to, pre-ictal 606a, ictal 606b, post-ictal 606c. The selected EEG data segments and corresponding clinical biomarker component labels are input to the seizure severity classifier for analysis in which the frequency, duration and raw signal characteristics of these periods/phases of the selected EEG data segments are then used to estimate a seizure severity score clinical biomarker based, by way of example only but not limited to, the Liverpool Seizure Severity Score.

The second ML model 607 is configured as a seizure severity classifier based on training a k-nearest neighbours ML technique using a labelled training dataset that includes data representative of features (particularly frequency-domain characteristics) extracted from, by way of example only but not limited to, N channels (e.g. 16 channels) of ictal EEG trace measurement data of test subjects during a trial. The ictal EEG trace measurement data of test subjects is collected in a controlled environment with experts assessing the clinical biomarker Liverpool Seizure Severity Score during the trial. These seizure severity scores, which is the clinical biomarker that is to be estimated by the seizure severity classifier, are then aligned with features extracted from windowed data and used as labelled training dataset for training the k-nearest neighbours ML technique for generating the seizure severity classifier. The trained seizure severity classifier is used as the second ML model 607 of the second processing stage 610.

In this example, the second ML model 607 receives input data based on EEG trace data segments classified with the clinical biomarker component labels associated with the bodily states of, by way of example only but is not limited to, pre-ictal 606a, ictal 606b, post-ictal 606c output by the first ML model 605a. For example, the EEG trace data segments may be 16 channels of EEG measurements that have been recorded from the subject and labelled as ictal signals by the first ML model 605a. The second ML model 607 processes the input data and outputs, by way of example only but not limited to, a Liverpool Seizure Severity Score clinical biomarker 608 of the subject.

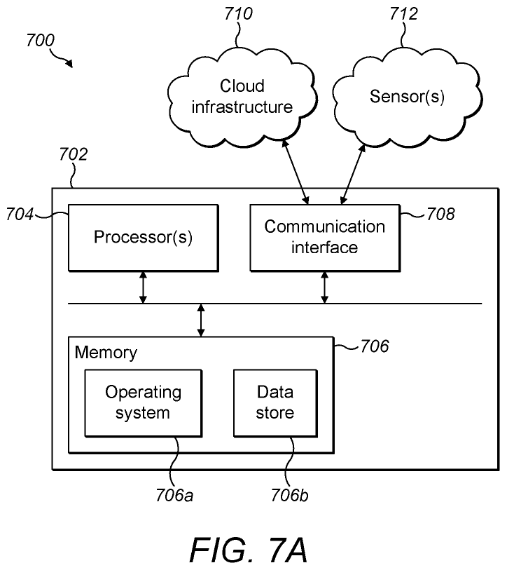

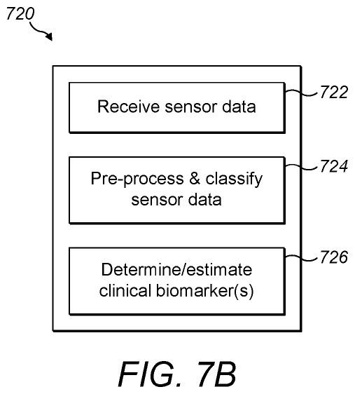

FIG. 7a is a schematic diagram of an example computing system 700 for estimating clinical biomarkers according to aspects of the invention. Computing system 700 may be used to implement one or more aspects of the systems, first and second processing stages, ML technique(s), first set of ML model(s) and/or second set of ML model(s), sensor pre-processing units and/or clinical biomarker processing units as described with as described with reference to FIGS. 1a-6b. Computing system 700 includes a computing device 702 that includes one or more processor unit(s) 704, memory unit 706 and communication interface 708 in which the one or more processor unit(s) 704 are connected to the memory unit 706 and the communication interface 708. The communications interface 708 may connect the computing device 702 with one or more sensors 712 associated with a subject, one or more device(s), one or more sensor(s), external or cloud storage or processing system(s) 710 and the like for implementing one or more aspects, features of the clinical biomarker estimation/detection system as described herein. The memory unit 706 may store one or more program instructions, code or components such as, by way of example only but not limited to, an operating system 706a for operating computing device 702 and a data store 706b for storing additional data, sensor data and the like, labelled training datasets, and/or further program instructions, code and/or components associated with implementing the functionality and/or one or more function(s) or functionality associated with one or more ML technique(s), labelling and/or training dataset(s) generation, one or more method(s) and/or process(es) of extracting and/or classifying one or more segments of sensor data, one or more of the method(s) and/or process(es) of estimation of clinical biomarkers, system(s)/platforms, combinations thereof, modifications there to, and/or as described herein with reference to at least one of figure(s) 1a to 6b.

FIG. 7b is a schematic diagram of another example system 720 for estimating clinical biomarkers according to a third embodiment. The system 720 for estimating one or more clinical biomarker(s) of a subject includes a communication interface 722 for receiving sensor data from one or more sensor(s) associated with the subject. The system 720 includes a sensor signal pre-processing unit 724 for extracting portions/segments of sensor data using a first set of one or more machine learning (ML) model(s) configured to extract and classify said portions/segments of the received sensor data based on clinical biomarker components associated with constructing and estimating one or more clinical biomarker(s) of the subject. The system 720 further includes a clinical biomarker estimation unit 726 for estimating one or more clinical biomarker(s) of the subject using a second set of one or more ML model(s) or mathematical model(s)/method(s) configured for estimating the one or more clinical biomarker(s) of the subject based on the extracted and classified portions/segments of sensor data. The communication interface 722, sensor signal pre-processing unit 724 and/or clinical biomarker estimation unit 726 may further include functionality associated with the one or more method(s), process(es), first and second processing stages, first set of ML model(s) and/or second set of ML model(s), combinations thereof, modifications thereto and/or as herein described with reference to any one of FIGS. 1a to 7a.

In the embodiment(s) described above the computing system, computing device, remote device, or systems above may include a server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1-4. (canceled)

5. A computer-implemented method for estimating one or more clinical biomarker(s) of a subject, the method comprising:
receiving sensor data from one or more sensor(s) associated with the subject;
processing the sensor data using a first set of machine learning (ML) model(s) configured to extract portions of sensor data relevant for estimating one or more clinical biomarker(s) of the subject;
receiving the extracted portions of sensor data from sensors associated with the subject; and
processing the received extracted portions of sensor data using a second set of ML model(s) configured to estimate the one or more clinical biomarker(s) of the subject.

6. The computer-implemented method as claimed in claim 5, wherein the sensor data comprises real-time sensor measurements of the subject.

7. (canceled)

8. The computer-implemented method as claimed in claim 5, wherein the sensor data comprises sensor measurements of the subject taken from a device recording data during administration of a treatment to the subject.

9. The computer-implemented method as claimed in claim 5, wherein the
sensor data comprises sensor measurements of the subject taken continuously whilst the subject performs their everyday activities, the method further comprising: pre-processing the sensor data using the first set of ML model(s) for extracting and classifying portions of the sensor data, and transmitting the extracted and classified portions of sensor data to a second computing device or unit for estimating, using the second set of ML model(s), one or more clinical biomarkers of interest present in the extracted and classified portions of sensor data.

10. The computer-implemented method as claimed in claim 5, wherein the portions of sensor data extracted by the first set of ML model(s) are further processed by one or more pre-processing algorithm(s) or further ML model(s) prior to inputting the extracted portions of sensor data to the second set of ML model(s) configured to estimate the one or more clinical biomarker(s) of the subject.

11. The computer-implemented method as claimed in claim 5, wherein a clinical biomarker comprises data representative of a metric or value that is calculated by a subject performing or undergoing a specific test in a clinical or laboratory environment that can be used as an indicator of a particular disease state or some other physiological state of a subject.

12. The computer-implemented method as claimed in claim 5, the method comprising constructing an estimate of the clinical biomarker based on:
receiving sensor data comprising sensor measurements taken of the subject whilst the subject performs their everyday activities;
extracting segments of the sensor data using the first set of ML model(s) to identify and classify each relevant segment of the sensor data based on one or more clinical biomarker components associated with the clinical biomarker;
constructing an estimate of the clinical biomarker based on inputting the extracted segments associated with the clinical biomarker components into one or more of the second set of ML model(s) for estimating the clinical biomarker.

13. The computer-implemented method as claimed in claim 12, wherein the second set of ML model(s) estimate a set of biomarker(s) and the step of constructing an estimate of the clinical biomarker further comprises estimating the clinical biomarker based on combining the set of biomarker(s) using a mathematical model and/or one or more ML model(s) of the second set of ML model(s) configured for estimating the clinical biomarker.

14. The computer-implemented method as claimed in claim 5, wherein the first set of machine learning (ML) model(s) are configured to classify the extracted portions of sensor data based on one or more clinical biomarker components associated with the one or more clinical biomarker(s).

15. The computer-implemented method as claimed in claim 14, wherein the second set of ML models are configured to estimate one or more clinical biomarkers of the subject based on receiving the extracted portions of sensor data and corresponding one or more clinical biomarker components as input.

16. (canceled)

17. The computer-implemented method as claimed in claim 5, wherein the sensor data is unstructured sensor data, the method further comprising inputting the unstructured sensor data to the first set of ML model(s) for extracting portions of unstructured sensor data relevant for estimating the one or more clinical biomarker(s)

18-49. (canceled)

50. A computer-implemented method for training a set of ML models for estimating one or more clinical biomarkers of a subject, the method comprising:
receiving a labelled sensor training dataset comprising extracted portions of sensor data classified in relation to clinical biomarker components and labelled with calculated clinical biomarkers of the subject;
inputting the labelled sensor training dataset to a set of ML technique(s) for generating one or more ML model(s) for estimating one or more clinical biomarker(s) of the subject based on the labelled sensor training dataset;
updating the ML technique(s) based on comparing the estimated one or more clinical biomarker(s) with the corresponding calculated clinical biomarkers of the subject;
repeating the inputting and updating steps until the ML techniques are determined to be validly trained;
outputting the corresponding trained set of ML model(s) configured for estimating clinical biomarkers based on sensor data segments classified to the corresponding clinical biomarker components.

51. The computer-implemented method as claimed in claim 50, wherein the labelled sensor training dataset is generated based on:
retrieving a first labelled sensor dataset of a test subject, the first labelled sensor dataset comprising sensor data segments classified based on a set of clinical biomarker components;
calculating one or more clinical biomarker(s) and/or one or more clinical biomarker components of the test subject required to be estimated using one or more ML models based on the first labelled sensor dataset of the test subject and corresponding clinical biomarker components;
labelling one or more segments of the first labelled sensor dataset with the corresponding calculated clinical biomarker(s); and
storing the labelled sensor training dataset for use in training one or more ML techniques to generate one or more ML model(s) configured to estimate one or more corresponding clinical biomarker(s) of interest from received extracted segments of sensor data, each of which have been classified based on one or more clinical biomarker component(s).

52. The computer-implemented method according to claim 50, wherein the method further comprises training one or more of the second set of ML model(s) for estimating one or more clinical biomarker(s) based on one or more other clinical biomarker(s) and/or associated extracted portions of sensor data.

53. The computer-implemented method according to claim 5, the method further comprising estimating a further clinical biomarker based on a combination of one or more of the estimated clinical biomarkers.

54-55. (canceled)

56. A system for estimating one or more clinical biomarker(s) of a subject, the system comprising:
a communication interface for receiving sensor data from one or more sensor(s) associated with the subject;
a sensor signal pre-processing unit for extracting portions of sensor data using a first set of machine learning (ML) model(s) configured to extract said portions of the received sensor data relevant for constructing and estimating one or more clinical biomarker(s) of the subject; and
a clinical biomarker estimation unit for estimating one or more clinical biomarker(s) of the subject using a second set of ML model(s) configured to estimate the one or more clinical biomarker(s) of the subject based on the extracted portions of sensor data.

57. The system as claimed in claim 56, wherein the first set of machine learning (ML) model(s) are configured to classify the extracted portions of sensor data based on one or more clinical biomarker components associated with constructing and estimating the one or more clinical biomarker(s).

58. The system as claimed in claim 56, wherein the second set of ML models are configured to construct an estimate one or more clinical biomarkers of the subject based on receiving the extracted portions of sensor data and corresponding one or more clinical biomarker components as input.

59. The system as claimed in claim 56, wherein the sensor data comprises real-time sensor measurements of the subject.

60. The system as claimed in claim 56, wherein one or more of the communication interface, the sensor signal pre-processing unit, or clinical biomarker estimation unit are configured to implement the corresponding steps of the computer-implemented method according to any of claims 1 to 54.

61-62. (canceled)
